(19) United States
(12) Patent Application Publication
(10) Pub. No.: US 2025/0019388 A1
(43) Pub. Date: Jan. 16, 2025

(54) STAT3 DEGRADERS AND USES THEREOF

(71) Applicant: Kymera Therapeutics, Inc., Watertown, MA (US)

(72) Inventors: Sagar Agarwal, Watertown, MA (US); Christopher De Savi, Watertown, MA (US); Joyoti Dey, Watertown, MA (US); Vaishali Dixit, Watertown, MA (US); Bradley Enerson, Watertown, MA (US); Ashwin Gollerkeri, Watertown, MA (US); Jared Gollob, Watertown, MA (US); Chris Ho, Watertown, MA (US); Michele Mayo, Watertown, MA (US); Haojing Rong, Watertown, MA (US); Bin Yang, Watertown, MA (US)

(21) Appl. No.: 18/894,810

(22) Filed: Sep. 24, 2024

Related U.S. Application Data
(62) Division of application No. 18/078,785, filed on Dec. 9, 2022, now abandoned.
(60) Provisional application No. 63/383,372, filed on Nov. 11, 2022.
(60) Provisional application No. 63/265,275, filed on Dec. 11, 2021.

(51) Int. Cl. C07F 9/30; A61K 9/00; A61P 35/00

(52) U.S. Cl. CPC C07F 9/302; A61K 9/0019; A61P 35/00

(57) ABSTRACT

The present invention relates to STAT3 degraders, their liquid formulations, and methods of use thereof for treating cancer.

### CROSS REFERENCE TO RELATED APPLICATIONS

This application is a divisional application of U.S. application Ser. No. 18/078,785, filed Dec. 9, 2022, which claims the benefit of priority to U.S. Provisional Appl. No. 63/265,275, filed Dec. 11, 2021, and U.S. Provisional Appl. No. 63/383,372, filed Nov. 11, 2022, the entirety of each of which is herein incorporated by reference.

### TECHNICAL FIELD OF THE INVENTION

The present invention relates to formulation and dosage forms of STAT3 degrader (2-(((5S,8S,10aR)-3-acetyl-8-(((S)-5-amino-1-(2-chloro-3-(4-(((S)-1-((2S, 4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl) amino)-4-oxobutyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid (Compound A), and methods of use thereof.

### BACKGROUND OF THE INVENTION

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases. UPP plays a key role in the degradation of short-lived and regulatory proteins important in a variety of basic cellular processes, including regulation of the cell cycle, modulation of cell surface receptors and ion channels, and antigen presentation.

The signal transducer and activator of transcription 3 (STAT3) protein is activated by cytokines and growth factors upon binding to their cognate cell surface receptors resulting in the recruitment and phosphorylation of STAT3 by Janus kinase (JAK), dimerization, nuclear translocation and transcriptional regulation of STAT3 target genes. While in normal cells STAT3 activity is tightly controlled by feedback regulation, in diseases including cancer and auto-immunity, STAT3 activity becomes deregulated by mechanisms that result in persistent activation of STAT3 as evidence by high levels of phosphorylated STAT3 (pSTAT3). Approximately 70% of human cancers including both hematological malignancies and solid tumors exhibit increased levels of pSTAT3. Aberrant activation of STAT3 has been shown to occur through direct mutation of the STAT3 gene, activation of upstream kinases such as JAK or ALK by mutation or translocation. reduced expression of negative regulators such as SOC3 and elevated receptor signaling from overexpression of cytokine and growth factors in the tumor microenvironment.

The mechanisms by which deregulated STAT3 contribute to tumor establishment and progression are multifactorial. Among the target genes regulated by STAT3 are key effectors of several hallmarks of cancer including proliferative signaling (CCND1, CCND2), resisting cell death (BCL2-L1, MCL-1), angiogenesis (VEGF, HIF1α), deregulated cellular energetics (MYC), avoiding immune destruction (PD-L1, IFNA) and tumor-promoting inflammation (IL-6). In cancer cell models with strong STAT3 activation such as anaplastic large cell lymphoma (ALCL), genetic knockdown of STAT3 is sufficient to inhibit proliferation and induce apoptosis confirming dependency on STAT3 signaling. In addition to these cancer cell autonomous pathway,. activated STAT3 also promotes a suppressive TME through direct regulation of immune cell function and regulation of cancer cell-TME crosstalk. Activation of STAT3 in innate and adaptive immune cells generally favors expansion of immune suppressive cells while reducing the proliferation, maturation and function of cytolytic effector cells. Targeting STAT3 with antisense oligonucleotides that are preferentially taken up by myeloid cells has been shown to reverse immune suppression and restore anti-tumor activity of cytotoxic T cells in mouse syngeneic tumor models. Finally STAT3 has been shown to be activated in response to both chemo- and targeted therapies such as EGFR inhibitors and contributes to the development of drug-resistance. Collectively these data illustrate the importance of STAT3 signaling to tumor establishment and growth, to tumor-extrinsic immune suppression in the TME and to the development of resistance to standard therapies thereby suggesting that selective degradation of STAT3 may be an effective means to suppress STAT3 signaling for the treatment of cancer.

A need exists to develop dosing and schedules for STAT3 degraders to improve upon the efficacy of STAT3 inhibitors and other therapies and provide single-agent activity in cancer therapy.

### SUMMARY OF THE INVENTION

It has been found that STAT3 degrader (2-(((5S,8S,10aR)-3-acetyl-8-(((S)-5-amino-1-(2-chloro-3-(4-(((S)-1-((2S, 4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl) phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid (Compound A), and its salts, formulations and unit dosage forms, as described herein, have certain advantages in treating hematological and solid tumors.

Accordingly, in one aspect, the present disclosure provides a liquid formulation or unit dosage form comprising Compound A, or a pharmaceutically acceptable salt thereof, and a pharmaceutically acceptable excipient and/or carrier. In some embodiments, a liquid formulation or unit dosage form of the invention comprises a sodium phosphate buffer. In some embodiments, a liquid formulation or unit dosage form of the invention is at about pH 6.5.

In another aspect, the present invention provides methods and uses for treating a hematological malignancy or solid tumor in a patient, comprising administering to the patient a therapeutically effect amount of compound A, or a pharmaceutically acceptable salt thereof, or a liquid formulation as described herein. In some embodiments, the hematological malignancy or solid tumor is a relapsed or refractory lymphoma. In some embodiments, the hematological malignancy or solid tumor is selected from large granular lymphocytic leukemia (LGL-L), peripheral T-cell lymphoma (PTCL), and cutaneous T-cell lymphoma (CTCL).

In some instances, the method comprises administering up to about 3.0 mg/kg of Compound A, or a pharmaceutically acceptable salt thereof, to the patient per day. In other instances, the method comprises administering up to about 500 mg of Compound A, or a pharmaceutically acceptable salt thereof, to the patient per day. In some embodiments, the method comprises administering Compound A, or a pharmaceutically acceptable salt thereof, to the patient intravenously. In some embodiments, the method comprises administering Compound A, or a pharmaceutically acceptable salt thereof, to the patient once per week (QW). In some embodiments, the method comprises administering Compound A, or a pharmaceutically acceptable salt thereof, to the patient on days 1, 8, 15, and 22 of a 28-day cycle.

In some embodiments, the present disclosure provides a compound, which is Compound A ammonium hydrogen salt.

These and other aspects of this disclosure will be apparent upon reference to the following detailed description. To this end, various references are set forth herein which describe in more detail certain background information and procedures and are each hereby incorporated by reference in their entirety.

### BRIEF DESCRIPTION OF THE DRAWINGS

FIG. 1A and FIG. 1B show the antitumor activity after QW and Q2W intravenous administration of Compound A in NOD SCID mice bearing SU-DHL-1 xenografts.

FIG. 2A and FIG. 2B show the antitumor activity after QW, 2 Days on/5 Days off, and Q2W intravenous administration of Compound A in NOD SCID mice bearing SUP-M2 xenografts.

FIG. 3 depicts a schematic of the drug product manufacturing process.

FIG. 4 depicts a schematic of the Phase 1 study design. *Solid tumor applies only to MTD/RP2D confirmation cohort. **RP2D not always the same as MTD.

FIG. 5 shows PK data from 4 patients enrolled in DL1.

FIG. 6 shows STAT3 degradation in blood at DL1.

### DETAILED DESCRIPTION OF CERTAIN EMBODIMENTS

### 1. General Description of Certain Embodiments of the Invention

Compound A is a potent, highly selective, intravenously administered, heterobifunctional small molecule therapeutic targeting the protein STAT3 and the E3 ligase von Hippel-Lindau protein (VHL) to mediate the selective degradation of STAT3 via the ubiquitin-proteasome system (UPS).

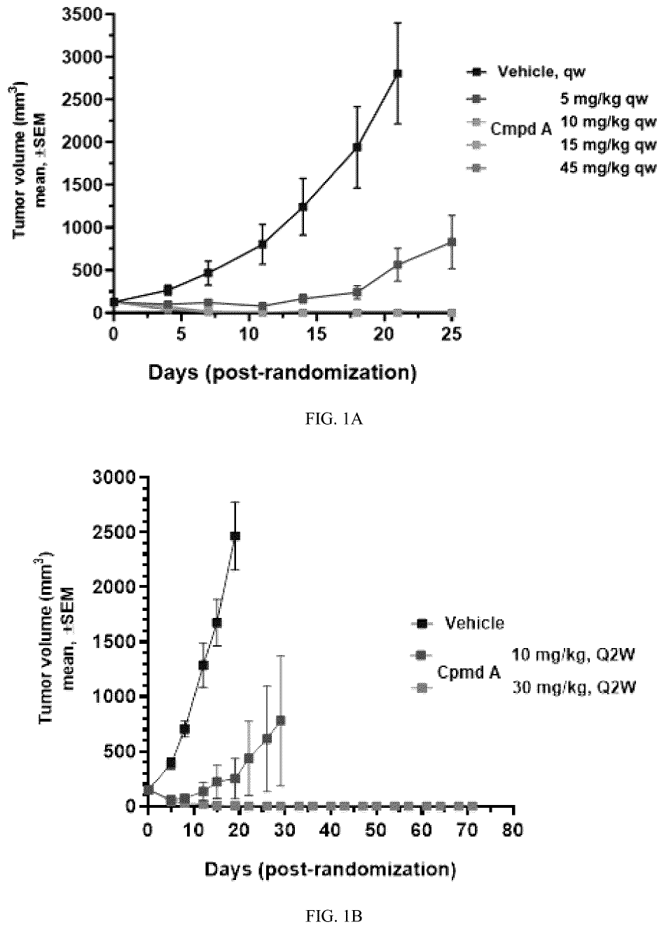

Compound A has demonstrated potent and selective STAT3 protein degradation and antitumor activity in a battery of in vitro and in vivo studies. In vitro, Compound A degrades STAT3 in human ALCL cell lines, SU-DHL-1 and SUP-M2, at low nanomolar range (≤11.8±2.3 nM), consistent with findings in the cellular phenotypic assays, where Compound A showed GI50 from 8.1 to 57.4 nM in several ALCL cell lines. Degradation of STAT3 in ALCL lines also induced caspase 3/7 activity, a marker of apoptosis, at similar concentrations. Washout experiments in the SU-DHL-1 cell line demonstrated irreversible growth inhibition, which occurred after approximately 48 hr of sustained degradation of STAT3. In SU-DHL-1 tumor xenograft murine model, 10 mg/kg QW dose of Compound A demonstrated significant antitumor efficacy with all mice in the treatment arm achieving complete regression (FIG. 1A). At this dose, greater than 90% STAT3 degradation in tumor was observed for 48 hrs. These data, combined with the result from the in vitro wash out study, suggest that a relatively short duration of STAT3 degradation is sufficient to induce an antitumor effect and support the potential for relatively short exposures and intermittent dosing regimens in clinic. Compound A exhibited comparable degradation potency of STAT3 in hepatocytes across human, rat, and dog. PK/PD study in rats also demonstrated significant degradation of STAT3 protein in multiple tissues following IV administration of Compound A. These data support the selection of rat and dog as nonclinical species for the safety evaluation of Compound A. In proteome wide assessment, Compound A is a highly selective STAT3 degrader that does not degrade other STAT family members or other cellular proteins expressed in peripheral blood mononuclear cell (PBMC).

Accordingly, in some embodiments, the present disclosure provides a method for treating a hematological malignancy or solid tumors in a patient, such as large granular lymphocytic leukemia (LGL-L), peripheral T-cell lymphoma (PTCL), and cutaneous T-cell lymphoma (CTCL), comprising administering to the patient a therapeutically effective amount of Compound A, or a pharmaceutically acceptable salt thereof, or a liquid formulation thereof as described herein.

In some embodiments, the present disclosure provides a method for treating a hematological malignancy in a patient, comprising administering to the patient a therapeutically effective amount of Compound A, or a pharmaceutically acceptable salt thereof, or a liquid formulation thereof as described herein.

In some embodiments, the present disclosure provides a method for treating relapsed or refractory lymphomas in a patient, comprising administering to the patient a therapeutically effective amount of Compound A, or a pharmaceutically acceptable salt thereof, or a liquid formulation thereof as described herein.

In some embodiments, the present disclosure provides a method for treating solid tumors in a patient, comprising administering to the patient a therapeutically effective amount of Compound A, or a pharmaceutically acceptable salt thereof, or a liquid formulation thereof as described herein.

In some embodiments, the present disclosure provides a method for treating LGL-L in a patient, comprising administering to the patient a therapeutically effective amount of Compound A, or a pharmaceutically acceptable salt thereof, or a liquid formulation thereof as described herein.

In some embodiments, the present disclosure provides a method for treating PTCL in a patient, comprising administering to the patient a therapeutically effective amount of Compound A, or a pharmaceutically acceptable salt thereof, or a liquid formulation thereof as described herein.

In some embodiments, the present disclosure provides a method for treating CTCL in a patient, comprising administering to the patient a therapeutically effective amount of Compound A. or a pharmaceutically acceptable salt thereof, or a liquid formulation thereof as described herein.

In some embodiments, the present disclosure provides a liquid formulation, which comprise Compound A, or a pharmaceutically acceptable salt thereof, and a pharmaceutically acceptable excipient and/or carrier. In some embodiments, the present disclosure provides a unit dosage form, which comprise Compound A, or a pharmaceutically acceptable salt thereof, and a pharmaceutically acceptable excipient and/or carrier.

In the following disclosure, certain specific details are set forth in order to provide a thorough understanding of various embodiments. However, one skilled in the art will understand that the methods and uses described herein may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof. such as. “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

### 2. Definitions

As used in the specification and appended claims, unless specified to the contrary, the following terms and abbreviations have the meaning indicated:

As used herein, the terms “about” or “approximately” have the meaning of within 20% of a given value or range. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

As used herein, “Compound A” refers to STAT3 degrader (2-(((5S,8S,10aR)-3-acetyl-8-(((S)-5-amino-1-(2-chloro-3-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl) phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid, of formula:

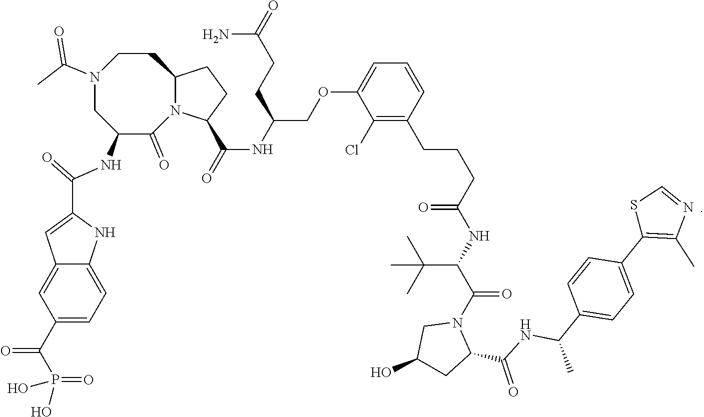 [SMILES: CC(=O)N1CC[C@H]2CC[C@@H](C(=O)N[C@@H](CCC(N)=O)COc3cccc(CCCC(=O)N[C@H](C(=O)N4C[C@H](O)C[C@H]4C(=O)N[C@@H](C)c4ccc(-c5scnc5C)cc4)C(C)(C)C)c3Cl)N2C(=O)[C@@H](NC(=O)c2cc3cc(C(=O)P(=O)(O)O)ccc3[nH]2)C1]

In some embodiments, Compound A is provided in solid form. In some embodiments, Compound A is amorphous.

As used herein, “Compound A ammonium hydrogen salt” (aka “Compound A ammonium salt”) refers to STAT3 degrader ammonium hydrogen (2-(((5S,8S,10aR)-3-acetyl-8-(((S)-5-amino-1-(2-chloro-3-(4-(((S)-1-((2S, 4R)-4-hydroxy-2-(((S)-1-(4-(4-methylthiazol-5-yl)phenyl)ethyl)carbamoyl) pyrrolidin-1-yl)-3,3-dimethyl-1-oxobutan-2-yl)amino)-4-oxobutyl)phenoxy)-5-oxopentan-2-yl) carbamoyl)-6-oxodecahydropyrrolo[1,2-a][1,5]diazocin-5-yl)carbamoyl)-1H-indole-5-carbonyl)phosphonate, of formula:

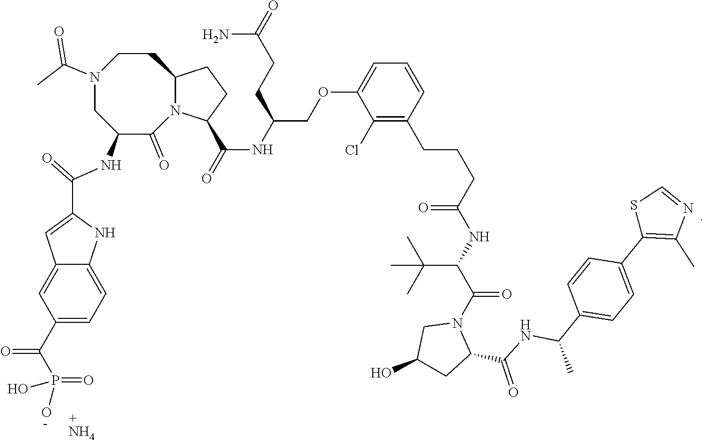

In some embodiments, Compound A ammonium hydrogen salt is provided in solid form. In some embodiments, Compound A ammonium hydrogen salt is amorphous.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N+(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The term “patient,” as used herein, means an animal, preferably a mammal, and most preferably a human. The term “subject,” as used herein, has the same meaning as the term “patient”.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, a patient or subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment or therapy.

A “therapeutically effective amount” or “therapeutically effective dosage” of a drug or therapeutic agent, such as Compound A, or a pharmaceutically acceptable salt thereof, is any amount of the drug that, when used alone or in combination with another therapeutic agent, protects a patient or subject against the onset of a disease, such as LGL-L, or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

In preferred embodiments, a therapeutically effective amount of the drug, such as Compound A, promotes regression to the point of eliminating the disease. In addition, the terms “effective” and “effectiveness” with regard to a treatment includes both pharmacological effectiveness and physiological safety. Pharmacological effectiveness refers to the ability of the Compound A, or a pharmaceutically acceptable salt thereof, to treat the disease in the patient. Physiological safety refers to the level of toxicity, or other adverse physiological effects at the cellular, organ and/or organism level (adverse effects) resulting from administration of the drug.

As used herein, the terms “therapeutic benefit” or “benefit from therapy” refers to an improvement in one or more of overall survival, progression-free survival, partial response, complete response, and overall response rate and can also include a decrease in severity of disease symptoms, an increase in frequency and duration of disease symptom-free periods, or a prevention of impairment or disability due to the disease affliction.

The phase “woman of childbearing potential” (WOCBP) are considered fertile: 1. following menarche; 2. from the time of menarche until becoming postmenopausal unless permanently sterile. A postmenopausal state is defined as no menses for 12 months without an alternative medical cause. A high follicle-stimulating hormone (FSH) level in the postmenopausal range may be used to confirm a postmenopausal state in women not using hormonal contraception or hormonal replacement therapy (HRT). However, in the absence of 12 months of amenorrhea, confirmation with more than one FSH measurement is required. Females on HRT and whose menopausal status is in doubt will be required to use one of the non-estrogen hormonal highly effective contraception methods if they wish to continue their HRT during the study. Otherwise, they must discontinue HRT to allow confirmation of postmenopausal status before study enrollment. Permanent sterilization methods (for the purpose of this study) include: documented hysterectomy; documented bilateral salpingectomy' documented bilateral oophorectomy; for individuals with permanent infertility due to an alternate medical cause other than the above, (e.g., Mullerian agenesis, androgen insensitivity, gonadal dysgenesis), Investigator discretion should be applied to determining study entry.

### 3. Description of Exemplary Embodiments

In some embodiments, the present invention provides a method for treating hematological and solid tumors in a patient, comprising administering to the patient a therapeutically effective amount of Compound A, or a pharmaceutically acceptable salt thereof, or a liquid formulation as described herein. In some embodiments, the hematological and solid tumors are relapsed and/or refractory lymphomas, large granular lymphocytic leukemia, and advance solid tumors. In some embodiments, the hematological and solid tumors are selected from large granular lymphocytic leukemia (LGL-L), peripheral T-cell lymphoma (PTCL), and cutaneous T-cell lymphoma (CTCL).

In some embodiments, the present disclosure provides a method for treating hematological and solid tumors in a patient, such as large granular lymphocytic leukemia (LGL-L), peripheral T-cell lymphoma (PTCL), and cutaneous T-cell lymphoma (CTCL), comprising administering to the patient a therapeutically effective amount of Compound A, or a pharmaceutically acceptable salt thereof, or a liquid formulation as described herein.

In some embodiments, the present disclosure provides a method for treating LGL-L in a patient, comprising administering to the patient a therapeutically effective amount of Compound A, or a pharmaceutically acceptable salt thereof, or a liquid formulation as described herein.

In some embodiments, the present disclosure provides a method for treating PTCL in a patient, comprising administering to the patient a therapeutically effective amount of Compound A, or a pharmaceutically acceptable salt thereof, or a liquid formulation as described herein.

In some embodiments, the present disclosure provides a method for treating CTCL in a patient, comprising administering to the patient a therapeutically effective amount of Compound A, or a pharmaceutically acceptable salt thereof, or a liquid formulation as described herein.

In some embodiments, the patient is male or female aged ≥18 years.

In some embodiments, the patient has histologically or pathologically confirmed Lymphomas (including Hodgkin's, B-cell, T-cell, Small Lymphocytic or NK-cell Lymphomas, or LGL-L) or solid tumors.

In some embodiments, the patient has histologically or pathologically confirmed PTCL, CTCL (WHO/EORTC Classification), LGL-L [T-cell LGL-L or Chronic Lymphoproliferative Disorder of NK-cells (CLPD-NK)], or solid tumors.

In some embodiments, fresh or archival formalin fixed paraffin embedded (FFPE) tumor tissue or 15 slides are preferably collected within ideally 6 months or 2 years prior to first dose of the study drug (for lymphoma and solid tumor patients, respectively). In some embodiments, when archival tissue/slides/blocks are not available, pre-dose biopsy will be performed (optional for Phase 1a, required for Phase 1b), and a blood sample collected during screening for STAT3 pathway mutational analysis and potentially for central pathology review.

In some embodiments, the lymphoma or solid tumor patient has relapsed and/or refractory disease to at least 2 prior systemic standard of care treatments or for whom standard therapies are not available.

In some embodiments, the LGL-L patient has relapsed and/or refractory disease to at least 1 prior systemic standard of care treatment or for whom standard therapies are not available.

In some embodiments, the patient of all disease types has relapsed and/or refractory disease to at least 1 prior systemic standard of care treatments or for whom standard therapies are not available.

In some embodiments, the LGL-L patient has hematology specific criteria selected from one of severe neutropenia <500/mm³, symptomatic anemia and/or, transfusion-dependent anemia; ANC≥200/μL at Screening and C1D1 (pre dose); or platelet count≥100.000/μL (assessed ≥7 days following last platelet transfusion in patients with thrombocytopenia requiring platelets).

In some embodiments, the LGL-L patient has baseline disease characteristics selected from CD3+CD8+cell population>650/mm³; CD3+CD8+CD57+population>500/mm³; presence of a clonal T-cell receptor (within 1 month of diagnosis); and Natural-Killer (NK) LGL is also permitted, provided there is a clonal NK-cell population noted with >500 cells/mm³.

In some embodiments, PTCL patients with solid tumors have measurable disease per Lugano for PTCL and Response evaluation criteria in solid tumors (RECIST) version 1.1 for solid tumors at Screening.

In some embodiments, patients have Eastern Cooperative Oncology Group (ECOG) performance status of 0-2 at Screening and C1D1 (pre-dose).

In some embodiments, patients have adequate bone marrow function at Screening and C1D1 (pre-dose) for all patients except those with LGL-L defined as: absolute neutrophil count (ANC)≥1000/μL; hemoglobin≥8 g/dL (for those patients undergoing red blood cell [RBC] transfusion, hemoglobin must be evaluated after at least 14 days after the last RBC transfusion); and platelet count≥100,000/μL (assessed ≥7 days following last platelet transfusion in patients with thrombocytopenia requiring platelets).

In some embodiments, patients with LGL-L have ANC≥200/μL at Screening and C1D1 (pre-dose).

In some embodiments, patients have adequate organ function at Screening and C1D1 (pre-dose) for all patients including those with LGL-L including aspartate aminotransferase (AST), alanine transaminase (ALT)≤3× upper limit of normal (ULN) or <5× ULN in cases of documented lymphoma involvement of liver; total serum bilirubin≤3× ULN or <5× ULN if secondary to Gilbert's syndrome or documented lymphoma involvement of liver; and serum creatinine clearance≥50 mL/min/1.73 m2 either measured or calculated using standard Cockcroft-Gault formula.

In some embodiments, female patients of childbearing potential (WOCBP) must agree to use highly effective contraceptive methods for the duration of treatment and 6 months after the last dose of Compound A. In some embodiments, WOCBP must have a negative serum pregnancy test at Screening and a negative serum or urine pregnancy test within 72 hours prior to first dose of Compound A.

In some embodiments, male patients must agree to use highly effective contraceptive methods during the treatment and for 6 months after the last dose of Compound A if the partner is a WOCBP.

In some embodiment the patient does not have a history or suspicion of central nervous system (CNS) metastases.

In some embodiment the patient does not have a diagnosis of Chronic Lymphocytic Leukemia (CLL).

In some embodiment the patient does not have a history of or active concurrent malignancy other than lymphoma or solid tumors unless the patient has been disease-free for >2 years. Exceptions to the ≥2-year time limit include treated basal cell or localized squamous cell skin carcinoma, localized prostate cancer, or other localized carcinomas such as carcinoma in situ of cervix, breast, or bladder.

In some embodiment the patient has not recovered from any clinically significant adverse events (AEs) of previous treatments to pre-treatment baseline or Grade 1 prior to first dose of Compound A.

In some embodiment the patient does not have ongoing unstable cardiovascular function: symptomatic ischemia, or uncontrolled clinically significant conduction abnormalities (i.e., ventricular tachycardia on antiarrhythmic drugs is excluded; 1st degree atrioventricular block or asymptomatic left anterior fascicular block/right bundle branch block will not be excluded), or congestive heart failure of New York Heart Association Class >III, or myocardial infarction within 3 months prior to Screening.

In some embodiment the patient does not have congenital long QT syndrome, or a QT interval corrected by Fridericia's formula (QTcF)≥450 ms (average of triplicate electrocardiograms) at Screening and/or on C1D1 (pre-dose) with the exception of a documented bundle branch block or unless secondary to pacemaker.

In some embodiment the patient does not have a history of thromboembolic or cerebrovascular event (i.e., transient ischemic attacks, cerebrovascular accidents, pulmonary emboli, or clinically significant deep vein thrombosis) within 2 years prior to screening.

In some embodiment the patient does not have an infection requiring antibiotics, antivirals, or antifungals within 1 week prior to first dose of Compound A. Prophylactic use of these agents is acceptable even if parenteral.

In some embodiment the patient does not have an active hepatitis B and/or hepatitis C infection as detected by positive hepatitis B surface antigen (HbsAg) or antibody to hepatitis C virus (anti HCV) with confirmation testing (e.g., anti-HBc, IgM anti-HBc, anti-HBs, HCV RNA), known seropositivity for human immunodeficiency virus (HIV).

In some embodiment the patient does not have a positive severe acute respiratory syndrome coronavirus-2 (SARS-CoV-2) test at Screening.

In some embodiment the patient does not have concurrent medical conditions including psychiatric disorders that in the judgment of the Investigator will interfere with the patient's ability to participate or with achieving the objectives of the study or pose a safety risk.

In some embodiment the patient is not pregnant or breast feeding.

In some embodiment the patient has not had an autologous hematopoietic stem cell transplant less than 3 months prior to first dose of Compound A.

In some embodiment the patient has not had prior allogenic hematopoietic or bone marrow transplant.

In some embodiment the patient has not had radiation treatment within 4 weeks prior to first dose of Compound A.

In some embodiment the patient has not had major surgery requiring general anesthesia within 4 weeks prior to first dose of Compound A.

In some embodiment the patient has not received a live vaccine within 1 month prior to the first dose of Compound A.

In some embodiment the patient has not had exposure to investigational or non-investigational anti-cancer therapy within 4 weeks or within at least 5 half-lives (up to a maximum of 4 weeks) prior to the first dose of Compound A, whichever is shorter. In all situations, the maximum washout period will not exceed 4 weeks prior to first dose of Compound A.

In some embodiment the patient has not completed a course of SARS-CoV-2 vaccine within 14 days prior to first dose of Compound A.

In some embodiment the patient has not used strong CYP3A4 inhibitors or inducers within 14 days or 5 half-lives of the first dose of Compound A (whichever is longer) within prior 14 days prior to first dose.).

In some embodiment the patient has not used OATP1B inhibitors or inducers within 14 days or 5 half-lives of the first dose of Compound A (whichever is longer) within prior 14 days prior to first dose.).

In some embodiment the patient has not used OATP1B, BCRP, and CYP2C8 substrates with narrow therapeutic index (as identified following discussion with medical monitor) within 14 days or 5 half-lives of the first dose of Compound A (whichever is longer) within prior 14 days prior to first dose).

In some embodiments, a method of the present invention comprises intravenously administering a liquid formulation as described herein. In some embodiments, a method of the present invention comprises administering a unit dosage form as described herein. In some embodiments, a method of the present invention comprises administering daily to a patient a liquid formulation or a unit dosage form as described herein.

### Liquid Formulations

According to one embodiment, the invention provides a liquid formulation comprising a STAT3 degrader of this invention (e.g., Compound A) or a pharmaceutically acceptable derivative thereof and a pharmaceutically acceptable excipient (e.g., a buffer) and/or carrier (e.g., water). The amount of Compound A in liquid formulations of this invention is such that it is effective to measurably degrade and/or inhibit STAT3 protein, or a mutant thereof, in a patient. In certain embodiments, a liquid formulation of this invention is formulated for administration to a patient in need of such composition. In some embodiments, a composition of this invention is formulated for parenteral (e.g., intravenous) administration to a patient.

In some embodiments, a liquid formulation of the invention comprises Compound A, or a pharmaceutically acceptable salt thereof (such as Compound A ammonium hydrogen salt), at a concentration of about 0.5%-1.5% w/w of the total weight of the formulation. In some embodiments, a liquid formulation of the invention comprises Compound A, or a pharmaceutically acceptable salt thereof (such as Compound A ammonium hydrogen salt), at a concentration of about 0.6%-1.4%, about 0.7%-1.3%, about 0.8%-1.2%, or about 0.9%-1.1% w/w of the total weight of the formulation. In some embodiments. a liquid formulation of the invention comprises Compound A, or a pharmaceutically acceptable salt thereof (such as Compound A ammonium hydrogen salt), at a concentration of about 0.60%, about 0.65%, about 0.70%, about 0.75%, about 0.80%, about 0.85%, about 0.9%, about 0.95%, about 1.00%, about 1.05%, about 1.10%, about 1.15%, about 1.20%, about 1.25%, about 1.30%, about 1.35%, about 1.40%, about 1.45%, or about 1.50% w/w of the total weight of the formulation. In some embodiments, a liquid formulation of the invention comprises Compound A at a concentration of about 0.995% w/w of the total weight of the formulation. In some embodiments, a liquid formulation of the invention comprises Compound A ammonium hydrogen salt at a concentration of about 1.00% w/w of the total weight of the formulation.

In some embodiments, a liquid formulation of the invention comprises Compound A, or a pharmaceutically acceptable salt thereof (such as Compound A ammonium hydrogen salt), at a concentration of about 5-15 mg/mL. In some embodiments, a liquid formulation of the invention comprises Compound A, or a pharmaceutically acceptable salt thereof (such as Compound A ammonium hydrogen salt), at a concentration of about 6-14 mg/mL, about 6.5-13.5 mg/mL, about 7-13 mg/mL, about 7.5-12.5 mg/mL, about 8-12 mg/mL, about 8.5-11.5 mg/mL, about 9-11 mg/mL, or about 9.5-10.5 mg/mL. In some embodiments, a liquid formulation of the invention comprises Compound A, or a pharmaceutically acceptable salt thereof (such as Compound A ammonium hydrogen salt), at a concentration of about 8 mg/mL, about 8.5 mg/mL, about 9 mg/mL, about 9.5 mg/mL, about 10 mg/mL, about 10.5 mg/mL, about 11 mg/mL, about 11.5 mg/mL, or about 12 mg/mL. In some embodiments, a liquid formulation of the invention comprises Compound A at a concentration of about 10 mg/mL. In some embodiments, a liquid formulation of the invention comprises Compound A ammonium hydrogen salt at a concentration of about 10.14 mg/mL.

The liquid formulation of the present invention may be administered parenterally by injection, infusion or implantation (intravenous, intramuscular, subcutaneous, or the like) as the liquid formulation or in unit dosage forms or via suitable delivery devices or implants containing conventional, non-toxic pharmaceutically acceptable carriers and adjuvants.

In some embodiments, a provided liquid formulation for parenteral use are provided in unit dosage forms (e.g., in single-dose ampoules), or in vials containing several doses and in which a suitable preservative may be added (see below). Typically, such compositions can be prepared as injectable formulations, for example, solutions or suspensions; solid and liquid forms suitable for using to prepare solutions or suspensions upon the addition of a reconstitution or dilution medium prior to injection; emulsions, such as water-in-oil (w/o) emulsions, oil-in-water (o/w) emulsions, and microemulsions thereof, liposomes, or emulsomes. In preferred embodiments, the liquid formulation or unit dosage forms thereof are administered intravenously. The preparation of such liquid formulations and unit dosage forms is described herein such as in Example 3.

If for intravenous administration, the liquid formulations or unit dose forms are packaged in solutions with one or more aqueous buffer. In some embodiments, the liquid formulations or unit dosage forms are packaged in solutions with sterile isotonic aqueous buffers. In some embodiments, the liquid formulations or unit dosage forms are buffered at about pH 5-8 or about pH 6-7 for parenteral administration upon dilution. In some embodiments, a buffering agent is at an amount to adjust the pH of a liquid formulation or a unit dosage form of the invention to about 6-8. In some embodiments, a provided liquid formulation or unit dosage form is at about pH 6.5. In some embodiments, a provided liquid formulation or unit dosage form is at pH 6.5±0.3. In some embodiments, a provided liquid formulation or unit dosage form is at about pH 6.0, about 6.1, about 6.2, about 6.3, about 6.4, about 6.5, about 6.6, about 6.7, about 6.8, about 6.9, or about 7.0. In some embodiments, the pH of a provided liquid formulation or unit dosage form can be adjusted by adding minute amounts of an acid (e.g., 1N hydrochloric acid) or a base (e.g., 1N sodium hydroxide).

Suitable buffers or buffering agents include, but are not limited to, phosphate buffers, citrate buffers, acetate buffers, histidine buffers, or succinate buffers. In some embodiments, the buffer is one or more phosphate buffer. In certain embodiments, the one or more phosphate buffer is disodium phosphate (e.g., disodium phosphate heptahydrate) and monobasic sodium phosphate (e.g., sodium phosphate monobasic monohydrate).

In some embodiments, a liquid formulation or unit dosage form of the invention comprises a sodium phosphate buffer. In some embodiments, a liquid formulation or unit dosage form of the invention comprises a sodium phosphate buffer at a concentration of about 25-75 mM, about 30-70 mM, about 35-65 mM, about 40-60 mM, or about 45-55 mM. In some embodiments. a liquid formulation or unit dosage form of the invention comprises a sodium phosphate buffer at a concentration of about 25 mM, about 30 mM, about 35 mM, about 40 mM, about 45 mM, about 50 mM, about 55 mM, about 60 mM, about 65 mM, about 70 mM, or about 75 mM. In some embodiments, a liquid formulation or unit dosage form of the invention comprises a sodium phosphate buffer at a concentration of about 50 mM.

In some embodiments, a liquid formulation or unit dosage form of the invention comprises a sodium phosphate buffer at a concentration of about 0.2%-1.1% w/w of the total weight of the formulation. In some embodiments, a liquid formulation or unit dosage form of the invention comprises a sodium phosphate buffer at a concentration of about 0.3%-1.0%, about 0.4%-0.9%, about 0.5%-0.8%, or about 0.6%-0.7% w/w of the total weight of the formulation. In some embodiments, a liquid formulation or unit dosage form of the invention comprises a sodium phosphate buffer at a concentration of about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1.0%, about 1.05%, or about 1.1% w/w of the total weight of the formulation. In some embodiments, a liquid formulation or unit dosage form of the invention comprises a sodium phosphate buffer at a concentration of about 0.64% w/w of the total weight of the formulation. In some embodiments, a liquid formulation or unit dosage form of the invention comprises a sodium phosphate buffer at a concentration of 0.636% w/w of the total weight of the formulation.

In some embodiments, a liquid formulation or unit dosage form of the invention comprises a sodium phosphate buffer at a concentration of about 2-11 mg/mL of the total weight of the formulation. In some embodiments, a liquid formulation or unit dosage form of the invention comprises a sodium phosphate buffer at a concentration of about 3-10, about 4-9, about 5-8, or about 6-7 mg/mL. In some embodiments, a liquid formulation or unit dosage form of the invention comprises a sodium phosphate buffer at a concentration of about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, or about 11 mg/mL. In some embodiments, a liquid formulation or unit dosage form of the invention comprises a sodium phosphate buffer at a concentration of about 6.4 mg/mL. In some embodiments, a liquid formulation or unit dosage form of the invention comprises a sodium phosphate buffer at a concentration of 6.36 mg/mL.

In some embodiments, the present invention provides a liquid formulation at about pH 6.5, comprising Compound A at a concentration of about 0.995% w/w of the total weight of the formulation, and a sodium phosphate buffer at a concentration of about 50 mM.

In some embodiments, the present invention provides a liquid formulation at about pH 6.5, comprising Compound A at a concentration of about 10 mg/mL, and a sodium phosphate buffer at a concentration of about 50 mM.

In some embodiments, the present invention provides a liquid formulation at about pH 6.5, comprising Compound A at a concentration of about 0.995% w/w of the total weight of the formulation, and a sodium phosphate buffer at a concentration of about 0.64% w/w of the total weight of the formulation.

In some embodiments, the present invention provides a liquid formulation at about pH 6.5, comprising Compound A at a concentration of about 10 mg/mL, and a sodium phosphate buffer at a concentration of about 0.64% w/w of the total weight of the formulation.

In some embodiments, the present invention provides a liquid formulation at about pH 6.5, comprising Compound A at a concentration of about 0.995% w/w of the total weight of the formulation, and a sodium phosphate buffer at a concentration of about 6.4 mg/mL.

In some embodiments, the present invention provides a liquid formulation at about pH 6.5, comprising Compound A at a concentration of about 10 mg/mL, and a sodium phosphate buffer at a concentration of about 6.4 mg/mL.

In some embodiments, the present invention provides a liquid formulation at about pH 6.5, comprising Compound A ammonium hydrogen salt at a concentration of about 1.00% w/w of the total weight of the formulation, and a sodium phosphate buffer at a concentration of about 50 mM.

In some embodiments, the present invention provides a liquid formulation at about pH 6.5, comprising Compound A ammonium hydrogen salt at a concentration of about 10.14 mg/mL, and a sodium phosphate buffer at a concentration of about 50 mM.

In some embodiments, the present invention provides a liquid formulation at about pH 6.5, comprising Compound A ammonium hydrogen salt at a concentration of about 1.00% w/w of the total weight of the formulation, and a sodium phosphate buffer at a concentration of about 0.64% w/w of the total weight of the formulation.

In some embodiments, the present invention provides a liquid formulation at about pH 6.5, comprising Compound A ammonium hydrogen salt at a concentration of about 10.14 mg/mL, and a sodium phosphate buffer at a concentration of about 0.64% w/w of the total weight of the formulation.

In some embodiments, the present invention provides a liquid formulation at about pH 6.5, comprising Compound A ammonium hydrogen salt at a concentration of about 1.00% w/w of the total weight of the formulation, and a sodium phosphate buffer at a concentration of about 6.4 mg/mL.

In some embodiments, the present invention provides a liquid formulation at about pH 6.5, comprising Compound A ammonium hydrogen salt at a concentration of about 10.14 mg/mL, and a sodium phosphate buffer at a concentration of about 6.4 mg/mL.

In some embodiments, the present invention provides a unit dosage form, which is a liquid formulation of the present invention, as described above, with a volume of about 10 mL. In some embodiments, the present invention provides a unit dosage form, which is a liquid formulation of the present invention, as described above, with a volume of about 10.5 mL. In some embodiments, the present invention provides a unit dosage form, which is a liquid formulation of the present invention, as described above, with a volume of about 10.1 mL, about 10.2 mL, about 10.3 mL, about 10.4 mL, about 10.6 mL, about 10.7 mL, about 10.8 mL, about 10.9 mL, about 11 mL, about 11.1 mL, about 11.2 mL, about 11.3 mL, about 11.4 mL, or about 11.5 mL.

In certain embodiments, the present invention provides a unit dosage form, which can be prepared by combining 101.4 mg Compound A ammonium hydrogen salt, 47.8 mg disodium phosphate heptahydrate, 44.1 mg sodium phosphate monobasic monohydrate, and water to about 10 mg/ml concentration of Compound A, and adding hydrochloride acid and sodium hydroxide to adjust pH to about 6.5.

In certain embodiments, the present invention provides a liquid formulation or a unit dosage form as described in the examples herein, such as Example 3. In certain embodiments, the present invention provides a liquid formulation or a unit dosage form, which can be prepared by the process as described in the examples herein, such as Example 3. In some embodiments, the unit dosage form comprises a liquid volume of about 10 mL.

Where necessary, the liquid formulation may also include a solubilizing agent. The components of the formulation can be either separately or mixed together in unit dosage form, for example, as a dry lyophilized powder (which can be reconstituted before use with a carrier such as saline) or concentrated solution in a hermetically sealed container such as an ampoule or sachet indicating the amount of active agent. If the composition is to be administered by infusion, it can be dispensed with an infusion bottle or bag containing sterile pharmaceutical grade water or saline. Where the formulation is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride. In preferred aspects, water is added to the formulation or unit dosage form of the present invention. In certain embodiments, the amount of water added to the formulation or unit dosage form is listed in Table 1 below.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to buffers, surfactants, dispersants, emulsifiers, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-laurylβiminodipropionate, myristoamphoacetate, lauryl betaine, and lauryl sulfobetaine. The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

In some embodiments, the liquid formulation or unit dose form of the present invention is mixed with an IV infusion vehicle. In some embodiments, the liquid formulation or unit dose form is mixed with an injectable medium such as normal saline (0.9% sodium chloride), 5% dextrose (D5W), or lactated ringer's injection. In some embodiments, the invention provides a liquid pharmaceutical composition prepared by mixing a liquid formulation or unit dose form of the invention with water, followed by dilution with saline or 5% dextrose. In some embodiments, a liquid pharmaceutical composition is diluted into a saline or 5% dextrose IV bag for IV administration. In some embodiments, a liquid pharmaceutical composition in a saline or 5% dextrose IV bag is stored under room temperature (about 20-25° C.) for up to about 4 hours before IV administration. In some embodiments, a liquid pharmaceutical composition in a saline or 5% dextrose IV bag is stored under refrigerated (about 2-8° C.) conditions for up to about 20 hours before IV administration. In some embodiments, a liquid pharmaceutical composition in a saline or 5% dextrose IV bag is stored under refrigerated (about 2-8° C.) conditions for up to about 20 hours, followed by storage under room temperature (about 20-25° C.) for up to about 4 hours, before IV administration.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular STAT3 degrader in the composition.

In some embodiments, the liquid formulation or unit dosage form of the present invention is a stabilized liquid formulation or a stabilized unit dosage form. In some embodiments, a liquid formulation or unit dosage form of the present invention is in frozen form. In some embodiments, the liquid formulation or unit dosage form of the present invention is stable after 3 freeze/thaw cycles. In some embodiments, the liquid formulation or unit dosage form of the present invention is stable for at least 3 months at 2-8° C. In some embodiments, the liquid formulation or unit dosage form of the present invention is stable for at least 12 months at −20° C. In some embodiments, the stability of the liquid formulation or unit dosage form of the present invention is shown in Example 4 below.

### Dosing and Schedules

As provided in view of preclinical data described herein, an STAT3 degrader (e.g., Compound A) or a pharmaceutically acceptable salt thereof, or a pharmaceutical composition thereof, is administered to a patient at a dose and schedule appropriate to give the desired cancer regression effect with minimum side effects. In some embodiments, a method of the present invention comprises administering daily to a patient up to about 3.0 mg/kg or up to about 5.0 mg/kg of Compound A (e.g., up to 201 mg or 350 mg for a 70 kg patient), for example about 0.25 mg/kg, about 0.5 mg/kg, about 0.75 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, or about 4.5 mg/kg of Compound A. In certain embodiments, the amount of Compound A administered daily to a patient is about 0.05, about 0.1, about 0.2, about 0.4, about 0.7, about 1.1, about 1.5, about 2.0, or about 2.7 mg/kg. In certain embodiments, the amount of Compound A administered daily to a patient is listed in Table 8 below.

In some embodiments, a method of the present invention comprises administering daily to a patient up to about 500 mg of Compound A, for example up to about 25 mg, up to about 50 mg, up to about 75 mg, up to about 100 mg, up to about 150 mg, up to about 200 mg, up to about 300 mg, up to about 400 mg, or up to about 500 mg of Compound A. In some embodiments, a method of the present invention comprises administering daily to a patient about 10-500 mg (for example, about 10-400 mg, about 50-400 mg, about 100-400 mg, about 200-400 mg, about 50-300 mg, about 100-300 mg, about 200-300 mg, about 25-200 mg, about 75-200 mg, about 100-200 mg, about 150-300 mg, or about 200-400 mg) of compound A. In some embodiments, a method of the present invention comprises administering daily to a patient about 50 mg of Compound A, for example 0.5×100 mg unit dosage forms. In some embodiments, a method of the present invention comprises administering daily to a patient about 100 mg of Compound A, for example 1×100 mg unit dosage forms. In some embodiments, a method of the present invention comprises administering daily to a patient about 150 mg of Compound A, for example 1.5×100 mg unit dosage forms. In some embodiments, a method of the present invention comprises administering daily to a patient about 200 mg of Compound A, for example 2×100 mg unit dosage forms. In some embodiments, a method of the present invention comprises administering daily to a patient about 250 mg of Compound A, for example 2.5×100 mg unit dosage forms. In some embodiments, a method of the present invention comprises administering daily to a patient about 300 mg of Compound A, for example 3×100 mg unit dosage forms. In some embodiments, a method of the present invention comprises administering daily to a patient about 350 mg of Compound A, for example 3.5×100 mg unit dosage forms. In some embodiments, a method of the present invention comprises administering daily to a patient about 400 mg of Compound A, for example 4×100 mg unit dosage forms. In some embodiments, a method of the present invention comprises administering a liquid formulation or a unit dosage form as described herein once daily. In some embodiments, a method of the present invention comprises administering a formulation or a unit dosage form as described herein twice daily. In some embodiments, a method of the present invention comprises administering a formulation or a unit dosage form as described herein three times daily. In some embodiments, a method of the present invention comprises administering a formulation or a unit dosage form as described herein four to fourteen times daily.

In some embodiments, where the patient is administered daily about 200 mg of Compound A, or a pharmaceutically acceptable salt thereof, the dosing is twice daily or BID, i.e., two separate about 100 mg doses. In some embodiments, where the patient is administered daily about 300 mg of Compound A, or a pharmaceutically acceptable salt thereof, the dosing is thrice daily or TID, i.e., three separate about 100 mg doses. In some embodiments, where the patient is administered daily about 400 mg of Compound A, or a pharmaceutically acceptable salt thereof, the dosing is four-times daily or QID, i.e., four separate about 100 mg doses.

In some embodiments, a method of the present invention comprises administering a liquid formulation or a unit dosage form as described herein, wherein there is about 4-24 hours between two consecutive administrations. In some embodiments, there is about 4, about 6, about 8, about 12, about 18, or about 24 hours between two consecutive administrations.

In some embodiments, a method of the present invention comprises administering a liquid formulation or a unit dosage form as described herein, wherein there are about 1-7 days between two consecutive administrations. In some embodiments, there are about 1, about 2, about 3, about 4, about 5, about 6, or about 7 days between two consecutive administrations. In certain embodiments, a liquid formulation or a unit dosage form as described herein is administered every 7 days between two consecutive administrations.

In some embodiments, a method of the present invention comprises administering a liquid formulation or a unit dosage form as described herein, wherein there is about 1-4 weeks between two consecutive administrations. In some embodiments, there is about 1, about 2, about 3, or about 4 weeks between two consecutive administrations. In some embodiments, a liquid formulation or a unit dosage form as described herein is administered once every two weeks (Q2W).

In some embodiments, Compound A is administered to a patient once every 1, 2, 3, 4, 5, 6, or 7 days. In some embodiments, a liquid formulation or a unit dosage form of the invention is administered to a patient biweekly (BIW). Biweekly doses can be administered hours apart (e.g., 1, 3, 6, 12 hours) or days apart (e.g., 1, 2, 3, or 4 days). In some embodiments, biweekly doses are administered on day 1 and day 2. In some embodiments, biweekly doses are administered on day 1 and day 4. In certain embodiments, a liquid formulation or a unit dosage form as described herein is administered once per week (QW). In some embodiments, Compound A is intravenously administered is administered to a patient once every 1, 2, 3, or 4 weeks, or once every 7, 10, 14, 17, 21, 24, or 28 days. In some embodiments, a liquid formulation or a unit dosage form as described herein is administered once every two weeks (Q2W).

As described herein in some embodiments, a liquid formulation or a unit dosage form is administered once weekly for two or three out of four weeks. In some embodiments, a liquid formulation or a unit dosage form as is administered twice weekly for two or three out of four weeks. In some embodiments, a liquid formulation or a unit dosage form is administered once weekly for two out of three weeks. In some embodiments, a liquid formulation or a unit dosage form is administered twice weekly for two out of three weeks. In some embodiments, a liquid formulation or a unit dosage form is administered once weekly every other week out of four weeks. In some embodiments, a liquid formulation or a unit dosage form is administered twice weekly every other week out of four weeks.

In some embodiments, a liquid formulation or a unit dosage form is administered to the patient once weekly in week 1 and week 2 in a 3 week administration cycle. In some embodiments, a liquid formulation or a unit dosage form is administered to the patient once weekly in week 1 and week 2 in a 4 week administration cycle. In some embodiments, a liquid formulation or a unit dosage form is administered to the patient once weekly in week 1 and week 2 in a 4 week administration cycle. In some embodiments, a liquid formulation or a unit dosage form is administered to the patient once weekly in week 1 and week 3 in a 4 week administration cycle. In some embodiments, a liquid formulation or a unit dosage form is administered to the patient once weekly in weeks 1-3 in a 4 week administration cycle. In some embodiments, a liquid formulation or a unit dosage form is administered to the patient once weekly in weeks 1-4 in a 4 week administration cycle (e.g., on days 1, 8, 15, and 22 of a 28-day cycle).

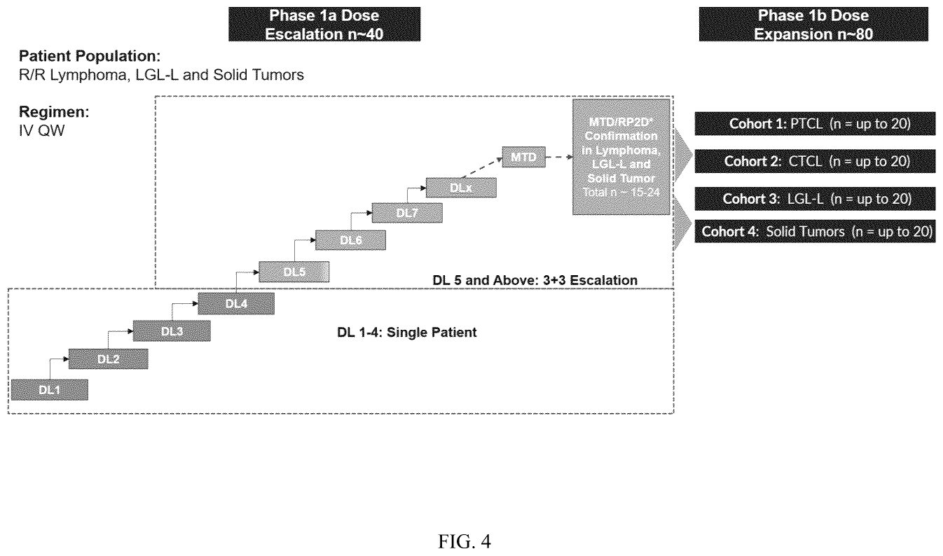

In some embodiments, a liquid formulation or a unit dosage form is administered to the patient twice weekly in week 1 and week 2 in a 3 week administration cycle. In some embodiments, a liquid formulation or a unit dosage form is administered to the patient twice weekly in week 1 and week 2 in a 4 week administration cycle. In some embodiments, a liquid formulation or a unit dosage form is administered to the patient once weekly in week 1 and week 2 in a 4 week administration cycle. In some embodiments, a liquid formulation or a unit dosage form is administered to the patient twice weekly in week 1 and week 3 in a 4 week administration cycle. In some embodiments, a liquid formulation or a unit dosage form is administered to the patient twice weekly in weeks 1-3 in a 4 week administration cycle. In some embodiments, the dosing schedule shown in FIG. 4.

In some embodiments, an IV infusion of a unit dosage form of the invention lasts about 5-180 minutes. In some embodiments, an IV infusion of a pharmaceutical composition of the invention lasts about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 minutes, or any range of time created by using two of the aforementioned times as endpoints. In some embodiments, an IV infusion of a unit dosage form of the invention lasts about 60-120 minutes. In some embodiments, an IV infusion of a unit dosage form of the invention lasts about 120-180 minutes. In some embodiments, an IV infusion of a unit dosage form of the invention lasts about 1, 2, 2.5, 3, 3.5, or 4 hours. In some embodiments, an IV infusion of a unit dosage form of the invention lasts about 2 hours.

### 4. Methods and Uses for Treating Disease

In some embodiments, the present invention provides a method for treating a hematological malignancy (e.g., such as various leukemias and lymphomas) or solid tumor in a patient, comprising administering to the patient a therapeutically effective amount of Compound A. In some embodiments, the hematological malignancy or solid tumor disease is large granular lymphocytic leukemia (LGL-L), peripheral T-cell lymphoma (PTCL), or cutaneous T-cell lymphoma (CTCL).

In some embodiments, the present disclosure provides a method for treating hematological malignancy in a patient, comprising administering to the patient a therapeutically effective amount of Compound A. In some embodiments, the hematological malignancy is leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenström's macroglobulinemia (WM), splenic marginal zone lymphoma, multiple myeloma, plasmacytoma, intravascular large B-cell lymphoma, AML, or MDS.

In some embodiments, the present disclosure provides a method for treating relapsed or refractory lymphomas in a patient, comprising administering to the patient a therapeutically effective amount of Compound A.

In some embodiments, the present disclosure provides a method for treating solid tumors in a patient, comprising administering to the patient a therapeutically effective amount of Compound A.

In some embodiments, the present disclosure provides a method for treating LGL-L in a patient, comprising administering to the patient a therapeutically effective amount of Compound A.

In some embodiments, the present disclosure provides a method for treating PTCL in a patient, comprising administering to the patient a therapeutically effective amount of Compound A.

In some embodiments, the present disclosure provides a method for treating CTCL in a patient, comprising administering to the patient a therapeutically effective amount of Compound A.

Without being limited to any particular theory, the present invention provides a method for treating of a proliferative disease selected from a benign or malignant tumor, solid tumor, liquid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkin's and Non-Hodgkin's, a mammary carcinoma, follicular carcinoma, undifferentiated carcinoma, papillary carcinoma, seminoma, melanoma, an IL-1 driven disorder, an MyD88 driven disorder, Smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenström's macroglobulinemia (WM), splenic marginal zone lymphoma, multiple myeloma, plasmacytoma, intravascular large B-cell lymphoma).

In some embodiments, the cancer which can be treated according to the methods of this invention is selected from glioma, breast cancer, prostate cancer, head and neck squamous cell carcinoma, skin melanomas, and ovarian cancer. In some embodiments, abnormal STAT3 activation also correlates with the progression of diverse hematopoietic malignancies, such as various leukemias and lymphomas, and STAT3 is frequently activated in both multiple myeloma cell lines and tumor cell lines derived from patient bone marrows.

In some embodiments, the present invention provides a method of treating a cancer selected from glioma, breast cancer, prostate cancer, head and neck squamous cell carcinoma, skin melanomas, ovarian cancer, malignant peripheral nerve shealth tumors (MPNST), pancreatic cancer, non-small cell lung cancer, urothelial cancer, liver cancer, bile duct cancer, kidney cancer, colon cancer, esophageal cancer, gastric cancer, gastrointestinal stromal tumors, and hematological malignancies include lymphomas, leukemias, myelomas, myeloproliferative neoplasms and myelodysplastic syndromes.

In some embodiments, the present invention provides a method of treating a JAK-associated disease. In some embodiments, the JAK-associated disease is cancer including those characterized by solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, uterine leiomyosarcoma, melanoma etc.), hematological cancers (e.g., lymphoma, leukemia such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides.

In some embodiments, the present invention provides a method of treating histologically or pathologically confirmed lymphomas (including Hodgkin's, B-cell, T-cell, Small Lymphocytic, or NK-cell Lymphomas). In some embodiments, the present invention provides a method of treating histologically or pathologically confirmed PTCL, CTCL, LGL-L [T-cell LGL-L or Chronic Lymphoproliferative Disorder of NK-cells (CLPD-NK)], or solid tumors.

### 5. Processes and Intermediates

In some embodiments, the present invention provides a process for preparing Compound A ammonium hydrogen salt. In some embodiments, the process for preparing Compound A ammonium hydrogen salt comprises treating intermediate F:

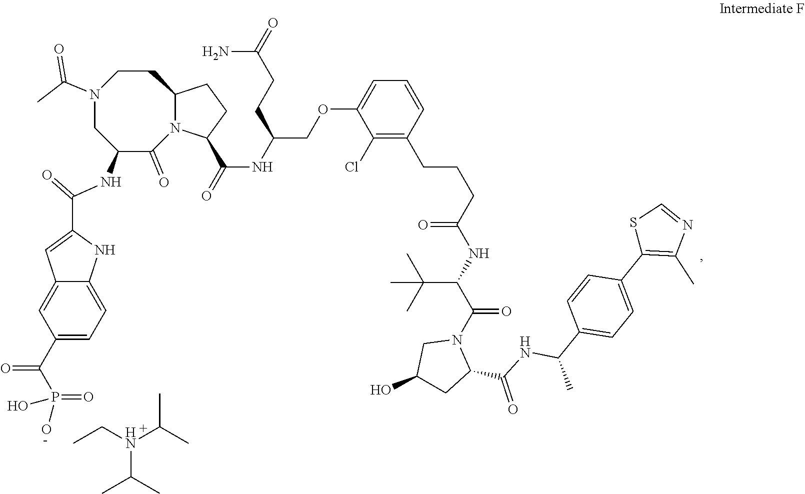

under suitable salt exchange conditions to form Compound A ammonium hydrogen salt.

In some embodiments, the suitable salt exchange conditions used to prepare Compound A ammonium hydrogen salt from intermediate F include conditions known in the art to exchange a DIPEA salt with an ammonium salt. In some embodiments, the suitable salt exchange conditions include subjecting intermediate F to an ammonium source, such as a solution containing ammonium hydrogen carbonate. In some embodiments, the suitable salt exchange conditions are described in the examples herein, such as Example 1.

In some embodiments, the process for preparing Compound A ammonium hydrogen salt further comprises preparing Intermediate F, the process comprising treating intermediate D:

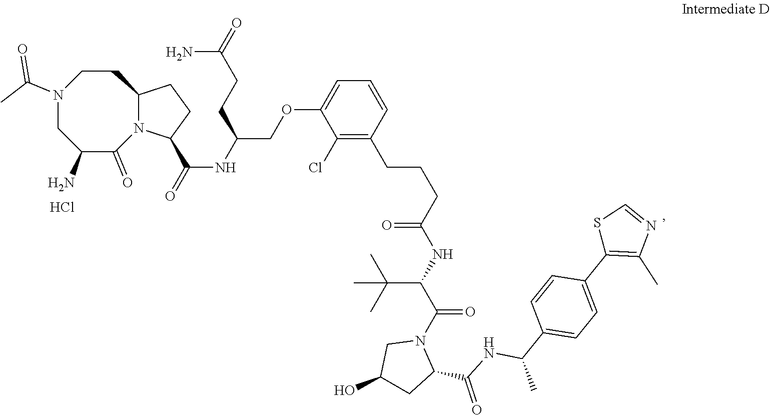 [SMILES: CC(=O)N1CC[C@H]2CC[C@@H](C(=O)N[C@@H](CCC(N)=O)COc3cccc(CCCC(=O)N[C@H](C(=O)N4C[C@H](O)C[C@H]4C(=O)N[C@@H](C)c4ccc(-c5scnc5C)cc4)C(C)(C)C)c3Cl)N2C(=O)[C@@H](N)C1 ; Cl]

with intermediate E:

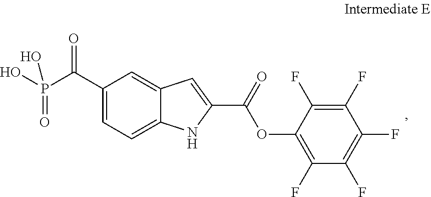 [SMILES: O=C(Oc1c(F)c(F)c(F)c(F)c1F)c1cc2cc(C(=O)P(=O)(O)O)ccc2[nH]1]

to form Intermediate F.

In some embodiments, the process for preparing intermediate F from intermediates D and F further comprises a base, such DIPEA. In some embodiments, the process for preparing intermediate F from intermediates D and F is described in the examples herein, such as Example 1.

In some intermediate embodiments, the present invention provides the DIPEA salt of Compound A (intermediate F):

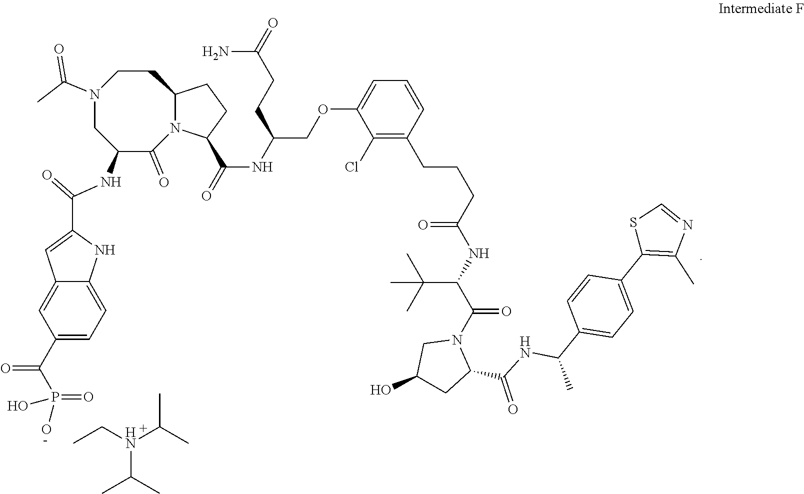

The following examples are provided for illustrative purposes only and are not to be construed as limiting this invention in any manner.

### EXEMPLIFICATION

List of Abbreviations
AE Adverse event
ALCL Anaplastic large cell lymphoma
ALT Alanine aminotransferase
ANC Absolute neutrophil count
AST Aspartate transaminase
AUC Area under the concentration-time curve
BSA Body surface area
CHOP Cyclophosphamide, doxorubicin, vincristine, and prednisone
CL Apparent total body clearance
Cmax Maximum plasma drug concentration
CNS Central nervous system
COVID-19 Coronavirus disease 2019
CR Complete response
CRBN Cereblon
eCRF Electronic case report form
CRO Contract research organization
CT Computed tomography
CTCL Cutaneous T-cell lymphoma
ctDNA Circulating tumor DNA
CYP Cytochrome P450
C1D1 Cycle 1 Day 1
C2D1 Cycle 2 Day 1
DCR Disease control rate
DDI Drug-drug interaction
DLT Dose-limiting toxicity
DNA Deoxyribonucleic acid
DOR Duration of response
DRF Dose range finding
ECG Electrocardiogram
ECOG Eastern Cooperative Oncology Group
EDC Electronic data capture
EMA European Medicine Agency
OI End of infusion
EOT End of treatment
FDA Food and Drug Administration
fe Fraction excreted/recovered in urine
FFPE Formalin-fixed paraffin-embedded
FFS Failure-free survival
FIH First-in-human
FSH Follicle-stimulating hormone
GCP Good Clinical Practice
GLP Good Laboratory Practice
HBcAb Hepatitis C core antibody
HBsAg Hepatitis B surface antigen
HCV Hepatitis C virus
HIV Human immunodeficiency virus
HRT Hormonal replacement therapy
IB Investigator's Brochure
ICF Informed consent form
ICH International Conference for Harmonisation
IEC Independent Ethics Committee
IMiD Immunomodulatory imide drug
INR International normalized ratio
IRB Institutional Review Board
IV Intravenous
JAK Janu kinase
LAR Legally authorized representative
LGL-L Large Granular Lymphocyte Leukemia
LHRH Luteinizing hormone-releasing hormone
MAD Maximum administered dose
MF Mycosis fungoides
MRI Magnetic resonance imaging
mSWAT Modified severity-weighted assessment tool
MTD Maximum tolerated dose
NCI TCAE National Cancer Institute Common Terminology Criteria for Adverse Events
NHL Non-Hodgkin Lymphoma
NK Natural killer
NTL Non-target Lesions
OR Objective response
ORR Objective response rate
OS Overall survival
PBMC Peripheral blood mononuclear cell
PD Pharmacodynamic(s)
PET Positron emission tomography
PFS Progression-free survival
PK Pharmacokinetic(s)
PR Partial response
PTCL Peripheral T-cell lymphoma
PTCL-NOS PTCL-not otherwise specified
q.s. Quantum sufficit (“as much as sufficient”)
QTcF QT interval corrected by Fridericia's formula
QW Once weekly
RBC Red blood cell
RECIST Response evaluation criteria in solid tumors
RP2D Recommended Phase 2 dose
R/R Relapsed/refractory
SAE Serious adverse event
SAP Statistical Analysis Plan

SARS-CoV-2 Severe acute respiratory syndrome coronavirus 2
SRC Safety Review Committee
SS Sézary syndrome
STAT Signal transducers and activators of transcription
SUSAR Suspected unexpected serious adverse reaction
t½ Elimination half-life
TEAE Treatment-emergent adverse event
TL Target lesion
tmax Time to reach Cmax following drug administration
LN Upper limit of normal
UPS Ubiquitin-proteosome system
US United States
Vd Apparent volume of distribution
Vdss Volume of distribution at steady state
WHO World Health Organization
WHODD World Health Organization Drug Dictionary
WOCBP Woman of childbearing potential

### Example 1. Synthesis of Compound A

Compound A can be prepared by methods known to one of ordinary skill in the art, for example, as described in WO 2020/206424, the contents of which, including below intermediates A, B, C, D, and E, are incorporated herein by reference in their entireties.

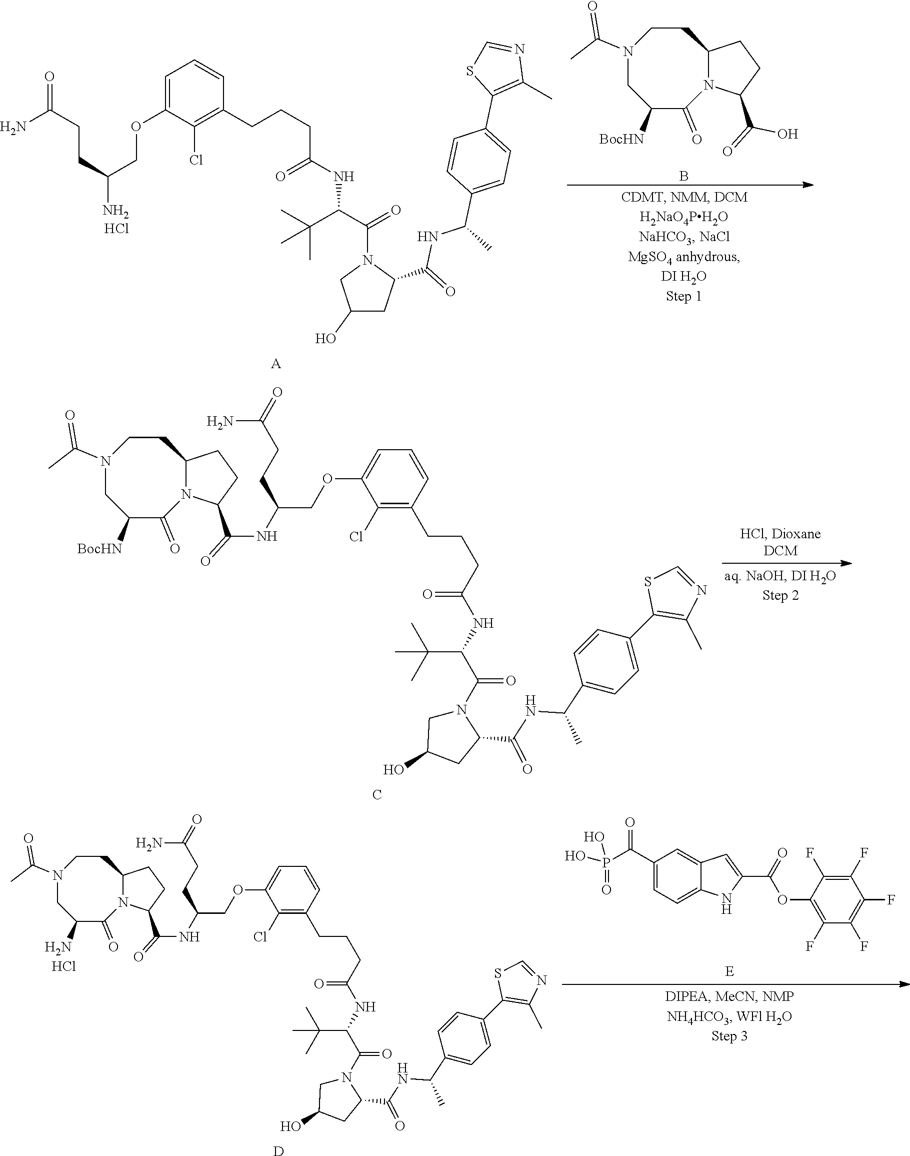

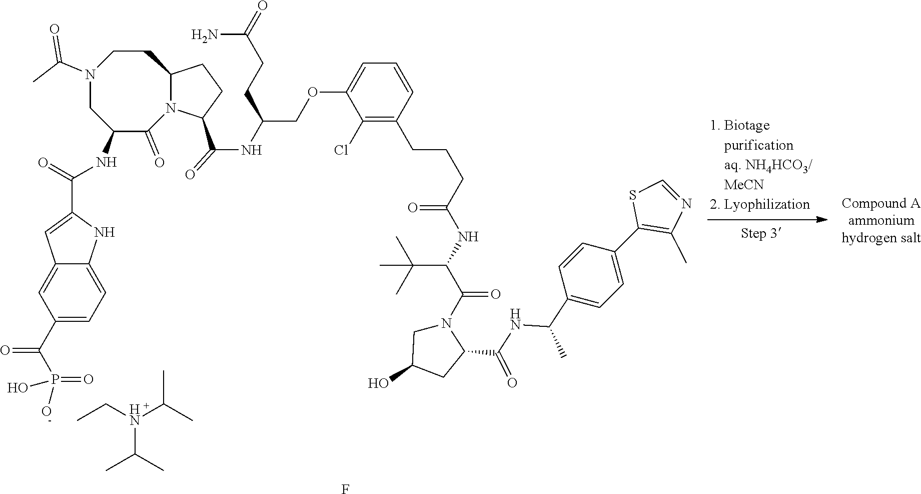

Step 1. Preparation of Intermediate C. To a room temperature solution of the amine A, the acid B and 2-chloro-4,6-dimethoxy-1,3,5-triazine (CDMT) in dichloromethane was added 4-methylmorpholine (NMM) slowly. The reaction mixture was stirred at this temperature until complete conversion of A to C is achieved (IPC, reaction conversion monitoring by HPLC). Upon reaction completion, deionized water was added. The layers were separated, and the organic phase was successively washed with aqueous solutions of sodium phosphate monobasic, sodium bicarbonate, and sodium chloride. The organic layer was dried over magnesium sulfate and the filtrate was tested for water content (IPC for water content by KF). The organic stream was concentrated down, and the resulting solution was used as is for step 2 (IPC, purity, and assay by HPLC).

Step 2. Preparation of Intermediate D. To a cold solution of the intermediate C in dichloromethane (DCM) was slowly added a solution of hydrochloric acid in dioxane. The reaction mixture was warmed to room temperature for at least 5 hours. Upon reaction completion (IPC, reaction monitoring by HPLC), the resulting solid was filtered, rinsed with DCM, and dried (IPC, water content by KF, purity by HPLC, and residual solvents by GC).

Step 3. Preparation of Intermediate F. To a cold suspension of the HCl salt of intermediate D in MeCN was added N,N-diisopropylethylamine (DIPEA) followed by a solution of intermediate E in N-methylpyrrolidinone (NMP) and a second charge of DIPEA. The reaction mixture was warmed to room temperature and stirred until complete conversion of intermediate D to F was achieved (IPC, reaction conversion monitoring by HPLC). The reaction mixture was transferred slowly to a room temperature solution of MeCN and the DIPEA salt of Compound A (intermediate F) precipitated. The suspension was then stirred at room temperature for at least 1 hour before filtration. The filtered solid was rinsed with MeCN and dried (IPC, purity by HPLC, residual solvents by GC).

Step 3′. Preparation of Compound A ammonium hydrogen salt. Intermediate F was purified by reverse phase preparative chromatography using ammonium bicarbonate and MeCN as the eluants (IPC, fraction purity by HPLC). The conforming fractions were combined and concentrated. The combined conforming fractions were concentrated (IPC, purity by HPLC) and lyophilized to yield Compound A ammonium hydrogen salt (IPC, water content by KF, residual solvent (MeCN) by GC, residual solvent (NMP and DIPEA) by GC).

### Example 2. In Vivo Xenograft Models

The antitumor efficacy of Compound A ammonium hydrogen salt was evaluated in immunocompromised mice implanted with human ALCL cell lines SU-DHL-1 and SUP-M2. Protocols for testing STAT3 degraders in human cell line xenograft models can be found, for example, in WO 2020/206424, incorporated herein by reference.

### SU-DHL-1

The antitumor activity of Compound A ammonium hydrogen salt was evaluated in a human SU-DHL-1 cell-line xenograft model established in NOD SCID female mice. Tumor bearing mice (n=5/group) were administered either 0 (vehicle; PBS), 5, 10, 15, or 45 mg/kg Compound A once weekly (QW; Days 0, 7, and 14) or 0 (vehicle; PBS), 10, or 30 mg/kg Compound A once every two weeks (Q2W; Days 0 and 14). Animals administered Compound A QW were monitored until Day 25 post first dose and those administered Compound A Q2W were monitored until Day 71 post first dose.

All animals in control groups (QW and Q2W) were euthanized on Days 25 and 19, respectively due to tumor burden. Overall, slight to moderate body weight increases were observed in animals administered Compound A by the IV route.

Compound A related anti-tumor activity was observed for all treatment groups in a dose-dependent manner. Animals administered IV doses of Compound A at 5 mg/kg QW achieved tumor growth inhibition (TGI) of 79.9%, while those administered 10, 15, or 45 mg/kg of Compound A QW all achieved complete regressions, which were sustained until study end (FIG. 1A).

Administration of Compound A Q2W at 10 and 30 mg/kg achieved 89.8 and 99.8% TGI, respectively, with all animals in the 30 mg/kg Q2W group achieving complete tumor regressions, which were sustained until study end (FIG. 1B). These data support the potential for intermittent dosing of Compound A by the IV route.

### SUP-M2

The antitumor activity of Compound A ammonium hydrogen salt was evaluated in a human SUP-M2 cell-line xenograft model established in NOD SCID female mice. Tumor bearing mice (n=5/group) were administered either 0 (vehicle; PBS), 10, 20, or 30 mg/kg Compound A QW (Days 0, 7, and 14), 10 or 20 mg/kg Compound A on a 2 days on/5 days off schedule (Days 0, 1, 7, 8, 14, and 15), or 0 (vehicle; PBS), 20, or 40 mg/kg Compound A Q2W (Days 0 and 14). Animals administered Compound A QW or 2 on/5 off were monitored until Day 25 post first dose, and those administered Compound A Q2W were monitored until Day 52 post first dose.

All animals in control groups (QW and Q2W) were euthanized early on Days 19 and 20, respectively, due to tumor burden. Overall, little to slight body weight increases were observed in animals administered Compound A by the IV route.

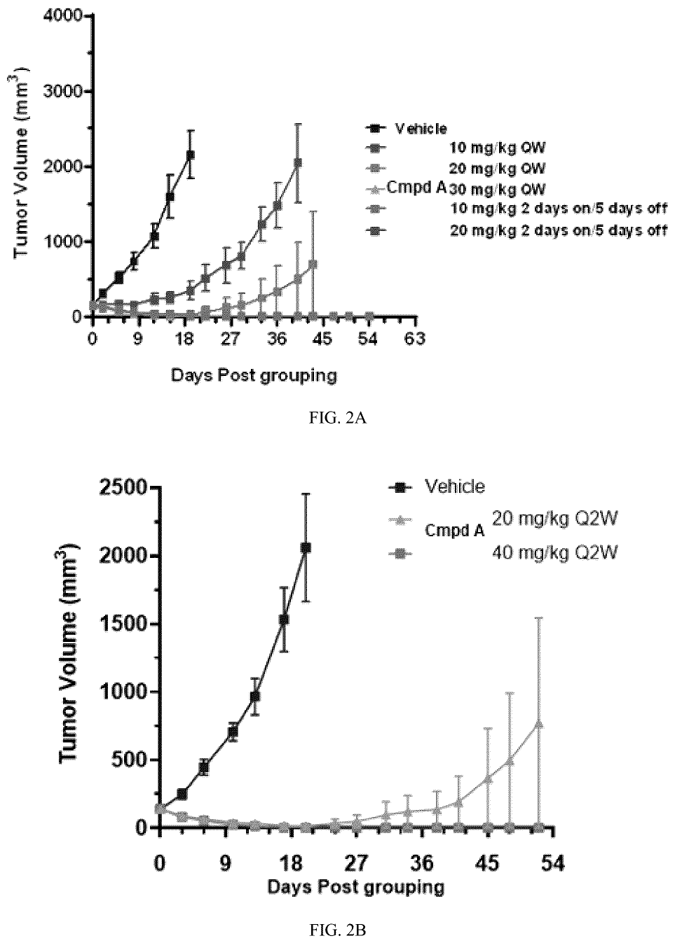

Compound A demonstrated significant, dose-dependent anti-tumor activity in SUP-M2 xenografts. Animals administered IV doses of Compound A at 10 mg/kg QW achieved TGI of 83.8%, while those administered 20 and 30 mg/kg QW achieved complete tumor regression in 4 of 5 and 5 of 5 animals, respectively, which was sustained until study end (FIG. 2A). Administration of Compound A according to a 2 day on/5 days off regimen at 10 and 20 mg/kg achieved complete tumor regression in all animals, which was sustained until study end (FIG. 2A). Q2W administration of Compound A at 20 and 40 mg/kg achieved complete tumor regression in 4 of 5 and 5 of 5 animals, respectively, which was sustained until study end (FIG. 2B).

### Example 3. Drug Product

The drug product, Compound A Injection (Concentrate Solution for Infusion), consists of a clear colorless solution of Compound A in clear Type I glass vials fitted with a rubber stopper and sealed with a flip-off aluminum cap. The drug product is formulated as 10 mg/mL of Compound A free acid (equivalent to 10.14 mg/mL of ammonium salt) dissolved in water for injection (WFI) containing disodium phosphate heptahydrate, and sodium phosphate monobasic monohydrate, adjusted to the target of pH 6.5 with either hydrochloric acid (HCl) or sodium hydroxide (NaOH).

The label fill volume is 10 mL. Each glass vial contains at a minimum 10.5 mL of sterile Compound A solution designed to deliver nominally 10.0 mL of the solution. The drug product solution is intended to be diluted to the required concentration with a diluent for intravenous infusion.

Quantitative composition of the drug product is given in Table 1.

TABLE 1
Composition of Compound A for Injection

| | | | Amount | Quality |
|---|---|---|---|---|
| Component | Function | % w/w | per 10 mL | Standard |
| Compound A | Active Ingredient | 0.995% ^{a} | 0.1000 g | In house |
| Disodium | Buffer | 0.478% | 0.0478 | USP |
| Phosphate | | | | |
| Heptahydrate | | | | |
| Sodium Phosphate | Buffer | 0.441% | 0.0441 | USP |
| Monobasic Monohydrate | | | | |
| 1N Hydrochloric Acid | pH adjustment | — | As needed | NF |
| 1N Sodium | pH adjustment | — | As needed | NF |
| Hydroxide | | | | |
| Water for Injection (WFI) | Solvent | q.s. to 100% | q.s. to 10 mL ^{b} | USP, EP, JP |

^{a} Based on free acid (1.000 mg Compound A free acid is equivalent to 1.014 mg Compound A hydrogen ammonium salt).
^{b} Minimal 0.5 mL overfill is included as per USP <1151> to ensure nominal withdrawal of 10.0 mL of solution.

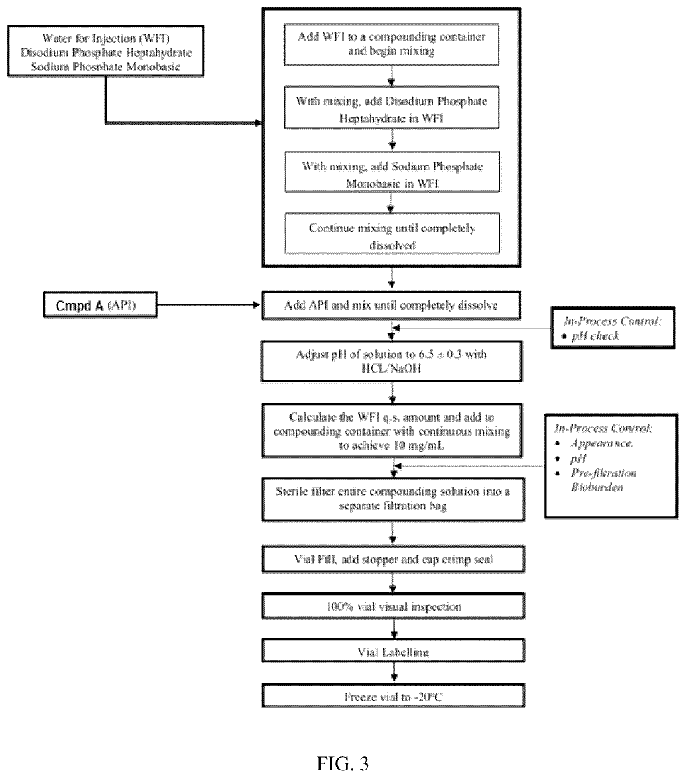

The drug product is manufactured by dissolving Compound A (off-white amorphous solid) into the solution of WFI, disodium phosphate heptahydrate and sodium phosphate monobasic monohydrate. The final pH is adjusted with HCl/NaOH to 6.5±0.3 and q.s. to 10 mg/ml with WFI. The solution is made in a 20 L glass vessel with stirring. The prepared solution is filtered through two sterilizing filters attached in series to obtain a sterile solution. The sterile solution is then filled into glass vials, stoppered, and crimped aseptically. Each vial is filled by weight to contain 10.5 mL of the sterile solution. The finished product is 100% visual inspected and labeling is performed. The vials are cooled to ensure uniform freezing at −20° C. A flow diagram of the manufacturing process is shown in FIG. 3.

### Example 4. Formulation Development

Compound A injection was manufactured as a frozen concentrated solution containing 10 mg/mL of free acid intended to be diluted with IV infusion vehicle. It has aqueous solubility greater than 10 mg/mL at pH 4.5 to 9.0 but less than 10 mg/mL at pH less than or equal to 3.

An R&D stability evaluation was conducted for the solution formulation containing 10 mg/mL of Compound A free acid dissolved in phosphate buffer at the pH range between 4.5 and 7.4. After 14 days stored at −20° C., 5° C., room temperature (RT), and 40° C., the assay and impurity results demonstrated that Compound A solution was chemically and physically stable at 5° C. and −20° C. Slight increase of impurities was noticed when it was stored at RT at pH 4.5. In addition, significant degradation was observed when Compound A solution was stored at 40° C. at pH 4.5 to 7.4.

Additional stability study was conducted on Compound A, 10 mg/mL solution, in phosphate buffer within a narrower pH range of 5.0 to 7.0 stored at −20° C., 5° C., room temperature, and 40° C. Chemical testing after 30 days indicated no significant growth of impurities when stored at −20° C., 5° C., and room temperature. However. significant impurity growth was detected at 40° C. within the pH 5.0 to 7.0. With these observations, the pH of the Compound A solution formulation for scale-up development was selected at 6.5±0.5 and the long-term storage condition was chosen at −20° C. as a frozen solution to ensure adequate long-term chemical and physical stabilities can be achieved.

A buffer screening study was performed as part of the development work. To demonstrate an acceptable short-term stability of Compound A solution, several 50 mM buffers were examined including phosphate (pH 6.5), citrate (pH 6.5), histidine (pH 6.5), and succinate (pH 6.0). 10 mg/mL of Compound A solution in different buffer type was stored at −20° C., 2-8° C., 25° C., and 40° C. After 14 days, the changes in assay and impurity profile are minimum for up to 25° C. and comparable across the evaluated buffers. However, significant color change was noted in histidine and succinate buffers when stored past 7 days at 40° C. storage condition. Impurities started to increase after 3 days at 40° C. for all evaluated buffers. Since satisfactory stability results were obtained on Compound A solution in phosphate buffer and no considerable advantage noted with other buffer types, the R&D scale development using 50 mM phosphate buffer at pH 6.5±0.5 was pursued.

The compatibility of Compound A at 10 mg/mL in 50 mM of phosphate buffer at pH 6.5 with three different IV infusion vehicles namely, normal saline (0.9% sodium chloride), 5% dextrose (D5W), and lactated ringer's injection were assessed. Based on the results of visual appearance, pH, assay, and impurity, normal saline was picked as a dilutant for IV infusion administration of Compound A solution.

Since the long-term storage of Compound A solution, 10 mg/mL, is at −20° C., a freeze-thaw stability was investigated for the frozen solution of Compound A solution, 10 mg/mL in 50 mM phosphate buffer at pH 6.5±0.5. The results illustrated that after a total of three freeze-thaw cycles, all vials showed no change in appearance, pH, assay, and impurities as compared to initial.

A R&D batch of Compound A, 10 mg/mL, solution in 50 mM of phosphate buffer (disodium phosphate and sodium phosphate monobasic) at pH 6.5±0.5 was made. Results obtained from the stability study of this R&D batch demonstrate that Compound A solution formulation was chemically and physically stable (i.e., Compound A solution formulation was clear, colorless, and free of visible particulates) for 18 months when stored at −20° C., 4 months at 2-8° C., and 2 months at 25° C.

Based on the formulation development work as discussed above, a 15.5-liter of GMP batch containing 10 mg/mL of Compound A free acid in 50 mM phosphate buffer at pH 6.5±0.5 was manufactured to support the first in human study.

A 12 month study was conducted on a sample of the GMP batch at −20° C. and stability and sterility was confirm at 12 months as shown in Table 2.

TABLE 2
12 Month Stability

| Test | Specification | T0 | 1 Month | 3 Months | 6 Months | 12 Months |
|---|---|---|---|---|---|---|
| Appearance | Clear/free of visual | Conforms | Conforms | Conforms | Conforms | Conforms |
| | particulate | | | | | |
| Assay by | Label Claim: | 98.9% | 102.6% | 104.9% | 103.6% | 104.2% |
| HPLC | 90-110% | | | | | |
| | Total Impurities | 1.9% | 2.0% | 2.3% | 2.2% | 2.3% |
| pH of | 6.50 ± 0.50 | 6.47 | 6.49 | 6.46 | 6.44 | 6.47 |
| Thawed | | | | | | |
| Particulate | ≥10 μm: ≤6000 | 20 | — | — | — | 153 |
| Matter | particles/vial | | | | | |
| | ≥25 μm: ≤600 | 2 | — | — | — | 1 |
| | particles/vial | | | | | |
| Bacterial | <0.18 EU/mg | <0.10 | — | — | — | <0.10 |
| Endotoxins | | | | | | |
| Sterility | Sterile | Sterile | — | — | — | No growth |

Container Closure System: The drug product was sterile filtered and filled into glass vials fitted with stopper with Flurotec barrier film secured by a flip-cap aluminum seal.

Microbiological Attribute: Drug product was manufactured aseptically and tested for Bacterial Endotoxin and Sterility for release and stability.

Compatibility: Frozen concentrated IV dosing solution stability/compatibility studies were conducted for Compound A Injection. These studies were designed to mimic the anticipated conditions, supplies, and procedures to be maintained during preparation of dosing solutions in the clinical setting. In clinic, each frozen of Compound A Injection 10 mg/mL is completely thawed at room temperature. Based upon the intended patient dose, the volume of thawed drug product solution is then calculated for addition to an appropriately sized normal saline IV bag. The final dilute dosing solution is then administered to the patient via a 1-2-hour IV infusion.

Compound A Injection Freeze-Thawed (FT) Study: To assess the stability of the thawed drug product, laboratory experiments were carried out under conditions representative of those found during clinical dose preparation. First each required vial of Compound A Injection was removed from −20° C. freezer and allowed to thaw under the room temperature laboratory lighting.

A freeze-thaw stability was investigated for the frozen Compound A Injection, 10 mg/mL. Four vials labelled as FT-T0, FT-1x, FT-2x, and FT-3x were allotted for the study. FT-T0 vial was tested initially and served as reference. The remaining three vials were subjected to freeze-thaw (FT) cycles. Each FT cycle involved freezing the drug product vial at −20° C. for 24 hours followed by the complete thawing at room temperature.

After 24 hours of freezing, all three vials (FT-1x, FT-2x, and FT-3x) were allowed to thaw at room temperature. FT-1x vial was pulled off for testing. The remaining two vials (FT-2x and FT-3x) were then subjected to second freezing again for 24 hours. Post-thawing, FT-2x was pulled off and tested for second freeze-thaw cycle. The remaining vial (FT-3x) was subjected to the final FT cycle. All the vials were tested for appearance, pH, assay, and impurities.

As presented in Table 3, Compound A Injection, 10 mg/mL, the results show acceptable physicochemical stability at least up to 3 FT cycles. All stability-indicating parameters of FT-1x, FT-2x and FT3x are comparable to the initial sample (FT-T0) and remained well within established drug product specification at each FT cycle.

TABLE 3
Freeze-Thaw Cycle Study Data for Compound A Injection, 10 mg/mL)

| | FT-TO | FT-1x | FT-2x | FT-3x |
|---|---|---|---|---|
| Appearance of | Clear, colorless | Clear, colorless | Clear, colorless | Clear, colorless |
| Thawed Drug | solution free of | solution free of | solution free of | solution free of |
| product | visible | visible | visible | visible |
| | particulates | particulates | particulates | particulates |
| pH | 6.46 | 6.47 | 6.43 | 6.48 |
| Assay (% LC) | 102.7 | 102.8 | 102.9 | 102.8 |
| Total Impurities (% w/w) | 2.3 | 2.2 | 2.3 | 2.3 |

Compound A Injection IV Dosing Solutions: To assess the stability and compatibility of Compound A Injection IV dosing solutions with the IV administration supplies (bag, tubing and close system transfer device) that are intended for use in the clinical trials, laboratory experiments were carried out under conditions representative of clinical dose preparation. The IV bag, administration sets (tubing), and close system transfer device (CSTD) that were utilized in this study are provided in Table 4.

TABLE 4
IV Administration Components Used in Stability and Compatibility
Studies of Compound A Injection Dosing Solutions

| IV | Minimal Requirements of | Example Component |
|---|---|---|
| Administration | Component Used in Clinical | Used in Stability/ |
| Component | Trial | Compatibility Study |
| Normal Saline IV | Meet USP specification for 0.9% | B. Braun Excel IV bags (Ethylene- |
| Infusion Bag | sodium chloride for injection | Propylene copolymer). Product code # |
| (500 cc) | PVC/DEHP-free, polyolefin bag | L8001 |
| Close System | Meet NIOSH guideline (DHHS | B. Braun On-Guard |
| Transfer | Publication No. 2004-165) | Vial adaptor (Product # 412111) |
| System (CSTD) | PVC/DEHP-free | Syringe adaptor (Product # 412118) |
| | | Spike port adaptor (Product # 412113) |
| | | General sterile luer lock syringe |
| | | (Becton Dickinson) |
| IV infusion set | No DEHP or natural rubber latex | B. Braun Infusomat space pump |
| | | IV administrative set (Product # |
| | | 490102) |
| | | Low absorption (Product # 490037) |
| Add on air- | No DEHP or natural rubber latex | B. Braun 1.2 μm air-eliminating filter |
| eliminating fdter | | (Product# 473994) |
| Catheter | PVC/DEHP-free | B. Braun Introcan safely catheter |
| | | (Product # 4251601-02) |

To accommodate a range of eventual clinical doses, studies were conducted at “bracketing” IV bag solution concentrations of 2 and 0.03 mg/mL (1000 and 15 mg equivalents). To prepare the IV dosing solutions, each required vial of Compound A for Injection (100 mg drug/10 mL) was thawed for at least one hour under ambient laboratory lighting and temperature conditions. Next the appropriate volume of drug product was calculated to achieve either 2 or 0.03 mg/mL (1000 and 15 mg equivalents) in the 500 cc IV bag. Prior to addition of the calculated volume of drug product, an equivalent volume of saline was first removed from the IV bag and discarded.

Once the drug product solution was added to the IV bag, the resulting dilute IV dosing solution was thoroughly mixed by hand and allowed to remain under ambient laboratory lighting and temperature conditions for the duration of the study. Samples were then pulled at 0, 8, 24 and 48 hours from the IV bag port via syringe. These samples were subsequently tested for stability-indicating parameters, including appearance, pH, and assay/impurity.

Meanwhile at time zero, for 2 mg/mL drug solution, the IV infusion set (tubing) with an air-eliminating filter extension and a catheter was connected to the IV bag and filled with the drug saline solution (primed). After the IV tubing was filled with the drug solution, put the catheter end of the IV tubing up to stop the drug solution flow, removed the IV tubing from the IV bag, held both end of the IV tubing up in a “U” position to retain the diluted drug saline solution inside the IV tubing and to allow full contact between the drug solution and IV tubing in the stationary stage as a worst-case scenario. At 8 hours, the drug solution in the IV tubing was then analyzed.

For the IV solution at 0.03 mg/mL concentration, the IV tubing with filter and catheter was flushed (filled and drained) 4 times with approximately 20 mL/flush of drug solution. For each flush fraction, the drug solution was collected and analyzed for assay to determine any assay drop of Compound A in the IV tubing. After the 4 flushes, the IV tubing was then filled with the drug solution, put the catheter end of the IV tubing up to stop the drug solution flow, removed the IV tubing from the IV bag, held both end of the IV tubing up in a “U” position to retain the diluted drug saline solution inside the IV tubing and to allow full contact between the drug solution and IV tubing in the stationary stage as a worst-case scenario. At 8 hours, the drug solution in the IV tubing was then analyzed.

Compound A Injection Dosing Solution (2 mg/mL) in IV Bag and infusion tubing: As presented in Table 5, IV dosing solutions at the concentration of 2 mg/mL prepared in IV bag and filled in IV tubing showed acceptable physicochemical stability and compatibility up to 48 hours and 8 hours, respectively. All stability-indicating parameters remained within established product specification at each time point and showed little to no change.

TABLE 5
IV Dosing Solution (2 mg/mL) Stability in IV Bag through 48 Hours and in IV Infusion
Tubing through 8 Hours under Ambient Storage Conditions

| | | | | | |
|---|---|---|---|---|---|
| Attribute | Sample Site | 0 hrs | 8 hrs | 24 hrs | 48 hrs |

Compound A Injection Dosing Solution: 2 mg/mL in IV bag

| | | | | | |
|---|---|---|---|---|---|
| Appearance- | IV bag port | Colorless | Colorless | Colorless | Colorless |
| color | | | | | |
| Appearance- | IV bag port | Clear and free | Clear and free | Clear and free | Clear and free |
| clarity | | of any visible | of any visible | of any visible | of any visible |
| | | particulate | particulate | particulate | particulate |
| pH | IV bag port | 6.30 | 6.29 | 6.34 | 6.31 |
| Assay (% LC) | IV bag port | 100 | 100 | 100 | 100 |
| Total | IV bag port | 2.0 | 2.0 | 2.0 | 2.0 |
| impurities | | | | | |

Compound A Injection Dosing Solution: 2 mg/mL in IV infusion tubing at 8 hours

| | | | | | |
|---|---|---|---|---|---|
| Appearance- | IV tubing | — | Colorless | — | — |
| color | | | | | |
| Appearance- | IV tubing | — | Clear and free | — | — |
| clarity | | | of any visible | | |
| | | | particulate | | |
| pH | IV tubing | — | 6.31 | — | — |
| Assay (% LC) | IV tubing | — | 99 | — | — |
| Total | IV tubing | — | 2.1 | — | — |
| impurities | | | | | |

Compound A Injection Dosing Solution (0.03 mg/mL) in IV Bag: As presented in Table 6, IV dosing solutions at the concentration of 0.03 mg/mL prepared in IV bag showed acceptable physicochemical stability and compatibility up to 48 hours. In the study of IV infusion tubing with filter and catheter, it appeared that the assay was dropped by 12% in the first flush fraction which indicated that Compound A was potentially adsorbed on the IV tubing after the first flush with 20 mL drug solution. However, % assay was closed 100% at 2 to 4 flushes as well as after 8 hours of drug solution holding in the IV administration set. These results indicated that amount of Compound A adsorbed on the IV administration set is minimum and only happens in the first 20 mL of drug solution at 0.03 mg/mL.

TABLE 6
Results of Assay in different Flush Fraction in the Same IV Administrative Set
Compound A Injection Dosing Solution: 0.03 mg/mL in IV infusion tubing

| | | 1st | 2nd | 3rd | 4th | |
|---|---|---|---|---|---|---|
| | Sample | Fill/drain | Fill/drain | Fill/drain | Fill/drain | |
| Attribute | Site | cycle | cycle | cycle | cycle | 8 hrs |
| Appearance- | IV tubing | — | — | — | — | Colorless |
| color | | | | | | |
| Appearance- | IV tubing | — | — | — | — | Clear and free |
| clarity | | | | | | of any visible |
| | | | | | | particulate |
| pH | IV tubing | — | — | — | — | 5.78 |
| Assay (% LC) | IV tubing | 88 | 103 | 103 | 103 | 100 |
| Total | IV tubing | — | — | — | — | — |
| impurities | | | | | | |

All stability-indicating parameters remained within established product specification at each time point and showed little to no change (Table 7). However, total impurities were not able to be determined since the impurity level in the diluted Compound A solution at 0.03 mg/mL was too low and below the detection limit of the release assay/impurity method.

TABLE 7
IV Dosing Solution (0.03 mg/mL) Stability in IV Bag through 48 Hours and in IV Infusion
Tubing through 8 hours under Ambient Storage Conditions
Compound A Injection Dosing Solution: 0.03 mg/mL in IV bag after each fill/drain cycle at 8 hrs

| Attribute | Sample Site | 0 hrs | 8 hrs | 24 hrs | 48 hrs |
|---|---|---|---|---|---|
| Appearance- | IV bag port | Colorless | Colorless | Colorless | Colorless |
| color | | | | | |
| Appearance- | IV bag port | Clear and free | Clear and free | Clear and free | Clear and free |
| clarity | | of any visible | of any visible | of any visible | of any visible |
| | | particulate | particulate | particulate | particulate |
| pH | IV bag port | 6.09 | 5.90 | 5.95 | 5.98 |
| Assay (% LC) | IV bag port | 100 | 99 | 99 | 99 |
| Total | IV bag port | — | — | — | — |
| impurities | | | | | |

Conclusions: The data from this study indicate that Compound A Injection frozen solution, 10 mg/mL, shows acceptable physicochemical stability after 3 cycles of freeze for 24 hours and completely thaw at room temperature. Likewise, simulated Compound A injection for IV dosing solutions at “bracketing” concentration of 0.03 and 2 mg/mL also show acceptable stability under ambient storage conditions in IV bag and IV fusion tubing for up to 48 hours and 8 hours, respectively. Moreover, the results from these studies suggest acceptable compatibility of Compound A injection with the intended diluent (0.9% normal saline) and containment/administration system (commercial IV bag/tubing) to be used in the clinical setting.

### Example 5. A Phase 1, Multicenter, Open-Label, Dose-Escalation and Expansion Study to Evaluate the Safety, Tolerability, Pharmacokinetics, Pharmacodynamics, and Clinical Activity of Intravenously Administered Compound A in Adult Patients with Relapsed or Refractory Lymphomas, Large Granular Lymphocytic Leukemia, and Solid Tumors

Rationale: Targeted protein degraders represent a new therapeutic class of compounds that utilize the ubiquitin proteasome system to target the specific degradation of proteins. Compound A is a protein degrader that targets signal transducers and activators of transcription (STAT)3, a transcription factor that plays an important role in hematological malignancies such as lymphomas and in solid tumors. Compound A is administered via intravenous infusion (IV) at the dose levels defined in the protocol on Days 1, 8, 15, and 22 of each 28-day cycle.

Objectives and Endpoints:

Phase 1a

| Objectives | Endpoints |
|---|---|
| Primary | |
| To evaluate the overall safety profile of escalating | Incidence and severity of adverse events (AEs) |
| doses of Compound A and to determine the | graded according to the National Cancer Institute |
| maximum tolerated dose (MTD) and the | (NCI) Common Terminology Criteria for Adverse |
| recommended Phase 2 dose (RP2D) in patients | Events (CTCAE), version 5.0, clinical laboratory |
| with relapsed/refractory (R/R) lymphoma and in | abnormalities, and electrocardiogram (ECG) |
| patients with advanced solid tumors | abnormalities |
| Secondary | |
| To characterize the pharmacokinetics (PK) of | Plasma and urine PK parameters for Compound A |
| Compound A in plasma and urine | |
| To obtain preliminary estimates of clinical activity | For R/R Lymphomas: Objective Response |
| of Compound A | Rate (ORR) based on Investigator’s |
| | assessment as per Lugano criteria 2014 and |
| | Duration of Response (DOR) |
| | For Cutaneous T-Cell Lymphoma (CTCL): |
| | Overall Response Rate by Modified Severity- |
| | Weighted Assessment Tool (mSWAT), DOR |
| | For solid tumors: RECIST 1.1 to determine |
| | ORR (based on Investigator’s assessment), |
| | complete response (CR), partial response |
| | (PR), DOR |
| | LGL-L: Determine ORR (by investigator |
| | assessment) including CR and PR, DOR |
| Exploratory | |
| To evaluate the relationship between the baseline | Comparison of clinical activity based on |
| mutational status of STAT3 and other relevant | mutational status in tumor and circulating tumor |
| genes and response to Compound A | DNA (ctDNA) |
| To assess the pharmacodynamic (PD) effects of | Change from baseline (pre-dose) in levels of PD |
| Compound A | biomarkers in both blood (peripheral blood |
| | mononuclear cells (PBMCs), plasma/serum) and |
| | tumor tissues |
| To evaluate the metabolite profile of Compound | Identification of potential metabolites in plasma |
| A in plasma and urine | and urine |

Phase 1b

| Objectives | Endpoints |
|---|---|
| Primary | |
| To evaluate the safety and tolerability of | Incidence and severity of AEs graded according |
| Compound A at the recommended Phase 2 dose | CTCAE, version 5.0, and changes in clinical |
| (RP2D) in patients with Peripheral T-cell | laboratory parameters, vital signs, and ECG |
| Lymphoma (PTCL), Large Granular Lymphocytic | abnormalities |
| Leukemia (LGL-L), CTCL, | |
| and solid tumors | |
| Secondary | |
| To obtain preliminary estimates of clinical | For relapsed/refractory (R/R) Lymphomas |
| activity of Compound A in adult patients with | and solid tumors: Objective Response Rate |
| PTCL, CTCL, LGL-L and solid tumors | (ORR), Duration of Response (DOR), |
| | Progression-free Survival (PFS), Disease |
| | control rate (DCR), and Overall Survival |
| | (OS) |
| | For CTCL: Overall Response Rate by |
| | Modified Severity-Weighted Assessment |
| | Tool (mSWAT), DOR |
| | For LGL-L: Determine ORR (by investigator |
| | assessment) including CR and PR, DOR |
| To characterize the pharmacokinetics (PK) of | Plasma and urine PK parameters for Compound A |
| Compound A in plasma and urine | |
| Exploratory | |
| To evaluate the relationship between the baseline | Comparison of clinical activity based on |
| mutational status of STAT3 and other relevant | mutational status in tumor and circulating tumor |
| genes and response to Compound A | DNA (ctDNA) |
| To assess the pharmacodynamic (PD) effects of | Change from baseline (pre-dose) in levels of PD |
| Compound A | biomarkers in both blood (peripheral blood |
| | mononuclear cells (PBMCs), plasma/serum) and |
| | tumor tissues |
| To evaluate the metabolite profile of Compound | Identification of potential metabolites in plasma |
| A in plasma and urine | and urine |

Overall Design: This is an open-label Phase 1a (dose escalation)/1b (dose expansion) first-in-human study of Compound A in adult patients with relapsed/refractory (R/R) lymphomas, LGL-L, or advanced solid tumors. The primary objective of the Phase 1a portion of the study is to identify the maximum tolerated dose [MTD]/recommended Phase 2 dose [RP2D]. Phase 1b will consist of separate cohorts of patients with R/R peripheral T-cell lymphoma (PTCL), cutaneous T-cell lymphoma (CTCL), large granular lymphocytic leukemia (LGL-L), and solid tumors.

Patients who provide informed consent and meet the eligibility criteria for the study will be enrolled and treated with Compound A IV on Days 1, 8, 15, and 22 of a 28-day cycle. Patients will remain on study treatment until disease progression, unacceptable toxicity, withdrawal of consent, any study-specific discontinuation criteria are met, or the Investigator determines that it is in the best interest of the patient to discontinue study treatment.

Fresh/archival FFPE tumor tissue will be obtained. When archival tissue/slides/blocks are not available, pre-dose biopsy will be performed (optional for Phase 1a, required for Phase 1b). One on-treatment biopsy will be required in Phase 1b unless medically contraindicated or is unattainable due to lack of feasibility. This biopsy will be optional in Phase 1a. An additional biopsy at time of disease progression will be optional for all patients. Any issues with collection of biopsies are to be discussed with medical monitor. The end of treatment/safety follow-up visit will be scheduled within 30 days from the last dose of Compound A and prior to initiation of a new anticancer therapy, whichever occurs first. Further, patients will be contacted every 3 months to collect data on survival status and subsequent therapies for up to one year after their last dose of Compound A.

Up to approximately 40 evaluable patients will be enrolled in Phase 1a; the total number of patients will depend on the number of dose levels explored. Up to 20 evaluable patients will be enrolled in each of the cohorts in Phase 1b. The study scheme is provided in FIG. 2.

Phase 1a: This part aims to characterize the safety and tolerability of IV weekly doses of Compound A in sequential cohorts. The dose escalation stage will be conducted in patients with R/R lymphoma, LGL-L or advanced tumors and will utilize an accelerated titration followed by a 3+3 design with the ultimate objectives of defining the maximum tolerated dose (MTD) and recommended Phase 2 dose (RP2D).

Following determination of MTD/RP2D, the dose will be confirmed prior to initiating enrollment in the respective cohorts (R/R lymphoma or LGL-L in Cohorts 1, 2, and 3, and advances solid tumors in Cohort 4) in Phase 1b.

Enrollment in phase 1b may not be initiated until the following criteria are met:
1) A total of at least 9 patients must have been treated at the MTD/RP2D, and
2) At least 6 patients with R/R lymphoma and advanced solid tumors, must have been treated at the MTD/RP2D for the respective Cohort(s) to begin enrolling in the Phase 1b.
3) A total of at least 3 LGL-L patients have been treated to begin enrolling in Phase 1b.

Enrollment in lymphoma, LGL-L, and solid tumor cohorts may be initiated independently as soon as criteria have been met for the respective cohorts.

Approximately 9 dose levels of Compound A are planned to be evaluated. The planned doses are shown in Table 8.

TABLE 8
Phase 1a Planned Dose Levels

| | Planned Dose ^{a} (mg/kg D1, |
|---|---|
| Dose Levels (DL) | 8, 15, 22 every 28 days) |
| 1 ^{b} (Starting dose) | 0.05 |
| 2 | 0.1 |
| 3 | 0.2 |
| 4 | 0.4 |
| 5 | 0.7 |
| 6 | 1.1 |
| 7 | 1.5 |
| 8 | 2.0 |
| 9 | 2.7 |

^{a} Planned dose levels are shown. Doses may be adjusted higher or lower based on emerging safety/PK/PD data from the study as determined by the SRC.
^{b} In case a dose reduction is required in the first cohort, a lower dose may be explored as recommended by the SRC.

The escalation dose levels and safety of dose escalation for ongoing patients will be determined by the Safety Review Committee (SRC) based on the review of all available data including, but not limited to safety and pharmacokinetic (PK).

Once MTD/RP2D is determined in 3-6 patients, it will be confirmed by enrolling additional R/R lymphoma, LGL-L, and advanced solid tumor patients (see above) until a total of 9 patients are enrolled prior to initiation of Phase 1b.

Phase 1b, Dose Expansion: After establishing the RP2D in patients with R/R lymphoma, LGL-1 and solid tumors, up to 80 additional patients will be treated to further characterize treatment-emergent adverse events (TEAEs) and to evaluate the relative clinical activity of Compound A in the following cohorts:
Cohort 1: PTCL (all subtypes of PTCL except CTCL) (n=up to 20).
Cohort 2: CTCL (n=up to 20)
Cohort 3: LGL-L (n=up to 20)
Cohort 4: Solid Tumors (n=up to 20)

Phase 1b expansion may start at separate times in Cohorts 1-3 and Cohort 4 and will be dependent on when the RP2D has been established in the R/R lymphoma, LGL-L and solid tumor confirmation portion of the Phase 1a. Patients will be treated at the RP2D as determined in the respective patient populations in Phase 1a. The starting dose in patients in Cohort 3 (LGL-L) will be the RP2D as determined in the lymphoma, LGL-L, and solid tumor patients in Phase 1a. If the DLTs in lymphoma across all patients are predominantly hematologic (i.e., neutropenia) or infectious in nature, a starting dose below the RP2D alternate regimens (e.g., IV every 2 weeks) or lower doses may be used evaluated in LGL-L patients following discussion with the SRC.

Patient's safety will be monitored throughout the study by the SRC established by the Sponsor. This committee will monitor all treatment-emergent data, e.g., PK and safety (including, but not limited to DLTs), on an ongoing basis to ensure the continued safety of patients enrolled in this study. Cumulative data will be monitored for any late onset toxicities.

### Study Population

### Inclusion Criteria

Patients are eligible to be included in the study only if all the following criteria apply:
1. Male or female aged ≥18 years on the day of signing the informed consent.
2. Patient understands signed and dated, written informed consent and provides voluntary consent prior to any mandatory study-specific procedures, sampling, and analyses. Patient is capable of giving signed informed consent which includes compliance with the requirements and restrictions listed in the informed consent form (ICF) and in this protocol.
3. Phase 1a Only: Histologically or pathologically confirmed Lymphomas including
   Hodgkin's,
   B-cell,
   T-cell,
   Small Lymphocytic, or NK-cell Lymphomas
   LGL-L (see inclusion #7, 9, 10)
   Histologically or pathologically confirmed solid tumors.
4. Phase 1b Only: Histologically or pathologically confirmed PTCL, CTCL (WHO/EORTC Classification), LGL-L [T-cell LGL-L or Chronic Lymphoproliferative Disorder of NK-cells (CLPD-NK)—see inclusion #7, 9, 10], or solid tumors.
5. Fresh or archival formalin fixed paraffin embedded (FFPE) tumor tissue or 15 slides preferably collected within ideally 6 months or 2 years prior to first dose of the study drug (for lymphoma and solid tumor patients, respectively). When archival tissue/slides/blocks are not available, pre-dose biopsy will be performed (optional for Phase 1a, required for Phase 1b), and a blood sample collected during screening for STAT3 pathway mutational analysis and potentially for central pathology review.
6. Phase 1a Only: Lymphoma and Solid Tumor: Relapsed and/or refractory disease to at least 2 prior systemic standard of care treatments or for whom standard therapies are not available.
7. Phase 1a: LGL-L: Relapsed and/or refractory disease to at least 1 prior systemic standard of care treatment or for whom standard therapies are not available.
8. Phase 1b Only: All disease types: Relapsed and/or refractory disease to at least 1 prior systemic standard of care treatments or for whom standard therapies are not available.
9. LGL-L Patients Only: (hematology specific criteria):
   One of the following:
      Severe neutropenia<500/mm³, or,
      Symptomatic anemia and/or,
      Transfusion-dependent anemia.
   ANC≥200/μL at Screening and C1D1 (pre dose)
   Platelet count≥100,000/μL (assessed ≥7 days following last platelet transfusion in patients with thrombocytopenia requiring platelets).
10. LGL-L Patients Only (baseline disease characteristics):
   CD3+CD8+cell population>650/mm³;
   CD3+CD8+CD57+population>500/mm³;
   Presence of a clonal T-cell receptor (within 1 month of diagnosis);
   Note: patients with T-LGLL may be included with PI approval even if CD3+CD8+cell population is <650/mm³ or CD3+CD8+CD57+population is <500/mm³, though +TCR is required;
   Natural-Killer (NK) LGL is also permitted, provided there is a clonal NK-cell population noted with >500 cells/mm³
11. PTCL and Solid Tumors Only: Measurable disease per Lugano for PTCL and Response evaluation criteria in solid tumors (RECIST) version 1.1 for solid tumors at Screening.
12. Eastern Cooperative Oncology Group (ECOG) performance status of 0-2 at Screening and C1D1 (pre-dose).
13. Adequate bone marrow function at Screening and C1D1 (pre-dose) for all patients except those with LGL-L defined as:
   Absolute neutrophil count (ANC)≥1000/μL.
   Hemoglobin≥8 g/dL (for those patients undergoing red blood cell [RBC] transfusion, hemoglobin must be evaluated after at least 14 days after the last RBC transfusion).
   Platelet count≥100,000/μL (assessed ≥7 days following last platelet transfusion in patients with thrombocytopenia requiring platelets).
14. Adequate organ function at Screening and C1D1 (pre-dose) for all patients including those with LGL-L.
   Aspartate aminotransferase (AST), alanine transaminase (ALT)≤3×upper limit of normal (ULN) or <5×ULN in cases of documented lymphoma involvement of liver.
   Total serum bilirubin≤3×ULN or <5×ULN if secondary to Gilbert's syndrome or documented lymphoma involvement of liver.
   Serum creatinine clearance≥50 mL/min/1.73 m2 either measured or calculated using standard Cockcroft-Gault formula.
15. Women of childbearing potential (WOCBP) must agree to use highly effective contraceptive methods for the duration of study treatment and 6 months after the last dose of Compound A.
16. WOCBP must have a negative serum pregnancy test at Screening and a negative serum or urine pregnancy test within 72 hours prior to first dose of the study drug.
17. Men must agree to use highly effective contraceptive methods during the study treatment and for 6 months after the last dose of study drug if the partner is a WOCBP.

### Exclusion Criteria

Patients are excluded from the study if any of the following criteria apply:
1. History or suspicion of central nervous system (CNS) metastases.
2. Diagnosis of Chronic Lymphocytic Leukemia (CLL).
3. History of or active concurrent malignancy other than lymphoma or solid tumors unless the patient has been disease-free for ≥2 years. Exceptions to the ≥2-year time limit include treated basal cell or localized squamous cell skin carcinoma, localized prostate cancer, or other localized carcinomas such as carcinoma in situ of cervix, breast, or bladder.
4. Patient has not recovered from any clinically significant adverse events (AEs) of previous treatments to pre-treatment baseline or Grade 1 prior to first dose of study drug.
5. Ongoing unstable cardiovascular function:
   Symptomatic ischemia, or.
   Uncontrolled clinically significant conduction abnormalities (i.e., ventricular tachycardia on antiarrhythmic drugs is excluded; 1st degree atrioventricular block or asymptomatic left anterior fascicular block/right bundle branch block will not be excluded), or
   Congestive heart failure of New York Heart Association Class≥III, or
   Myocardial infarction within 3 months prior to Screening.
6. Congenital long QT syndrome, or a QT interval corrected by Fridericia's formula (QTcF)≥450 ms (average of triplicate electrocardiograms) at Screening and/or on C1D1 (pre-dose) with the exception of a documented bundle branch block or unless secondary to pacemaker. In the case of a documented bundle branch block or a pacemaker, discussion with the Medical Monitor is required prior to enrollment.
7. History of thromboembolic or cerebrovascular event (i.e., transient ischemic attacks, cerebrovascular accidents, pulmonary emboli, or clinically significant deep vein thrombosis) within 2 years prior to screening.
8. Infection requiring antibiotics, antivirals, or antifungals within 1 week prior to first dose of study drug. Prophylactic use of these agents is acceptable even if parenteral.
9. Active hepatitis B and/or hepatitis C infection as detected by positive hepatitis B surface antigen (HbsAg) or antibody to hepatitis C virus (anti HCV) with confirmation testing (e.g., anti-HBc, IgM anti-HBc, anti-HBs, HCV RNA), known seropositivity for human immunodeficiency virus (HIV).
10. Positive severe acute respiratory syndrome coronavirus-2 (SARS-CoV-2) test at Screening.
11. Concurrent medical conditions including psychiatric disorders that in the judgment of the Investigator will interfere with the patient's ability to participate or with achieving the objectives of the study or pose a safety risk.
12. Patient is pregnant or breast feeding.
13. Autologous hematopoietic stem cell transplant less than 3 months prior to first dose of study drug.
14. Prior allogenic hematopoietic or bone marrow transplant.
15. Radiation treatment within 4 weeks prior to first dose of study drug.
16. Major surgery requiring general anesthesia within 4 weeks prior to first dose of study drug. If patient required general anesthesia within the prior 4 weeks, consultation with the Medical Monitor is required prior to enrollment.
17. Received live vaccine within 1 month prior to the first dose of study drug.
18. Exposure to investigational or non-investigational anti-cancer therapy within 4 weeks or within at least 5 half-lives (up to a maximum of 4 weeks) prior to the first dose of study drug, whichever is shorter. In all situations, the maximum washout period will not exceed 4 weeks prior to first dose of study drug. Note: Low dose steroids (oral prednisone or equivalent≤20 mg/day), localized non-CNS radiotherapy, previous hormonal therapy with luteinizing hormone-releasing hormone agonists for prostate cancer, and treatment with bisphosphonates and RANKL inhibitors are not criteria for exclusion.
18. Patient has completed a course of SARS-CoV-2 vaccine within 14 days prior to first dose of study drug.
19. Use of strong CYP3A4 inhibitors or inducers within 14 days or 5 half-lives of the first dose of study drug (whichever is longer) within prior 14 days prior to first dose.).
20. Use of OATP1B inhibitors or inducers within 14 days or 5 half-lives of the first dose of study drug (whichever is longer) within prior 14 days prior to first dose.).
21. Use of OATP1B, BCRP, and CYP2C8 substrates with narrow therapeutic index (as identified following discussion with medical monitor) within 14 days or 5 half-lives of the first dose of study drug (whichever is longer) within prior 14 days prior to first dose.).
22. Patient is unable or unwilling to discontinue prohibited concomitant medications or adhere to restrictions for use of concomitant medications.
23. Patient is unable or unwilling to comply with all requirements of the study.
24. Person who has been committed to an institution by official or judicial order.
25. Sponsor or Investigator site staff who are directly involved in the conduct of the study, site staff otherwise supervised by the Investigator, and their respective family members.

### Statistical Considerations

No formal statistical hypotheses will be tested in this dose escalation and dose expansion, single treatment group study. Safety, efficacy, PK, and pharmacodynamics assessments will be summarized separately for the dose escalation and dose expansion portions of the study. Additional summaries of pooled data across dose levels and/or Expansion cohorts may also be generated. Descriptive and summary statistics will be presented for the assessments and will include number of observations, mean, standard deviation, median, and range for continuous variables while categorical data will be summarized using frequency counts and percentages. Listings and graphical summaries of the data may be presented. All details of the data summaries and displays will be presented in a formal Statistical Analysis Plan which will be finalized prior to final database lock.

### Preliminary Results

STAT3 degradation in blood at first dose level was consistent with preclinical predictions, with mean maximum degradation following first 2 doses of Cycle 1 averaging 66%, with maximum knockdown of up to 86%. At least 72 h of target degradation observed that in preclinical species led to robust antitumor activity in STAT3 sensitive models.

DL1 level was safe and well-tolerated with no DLTs or SAEs.

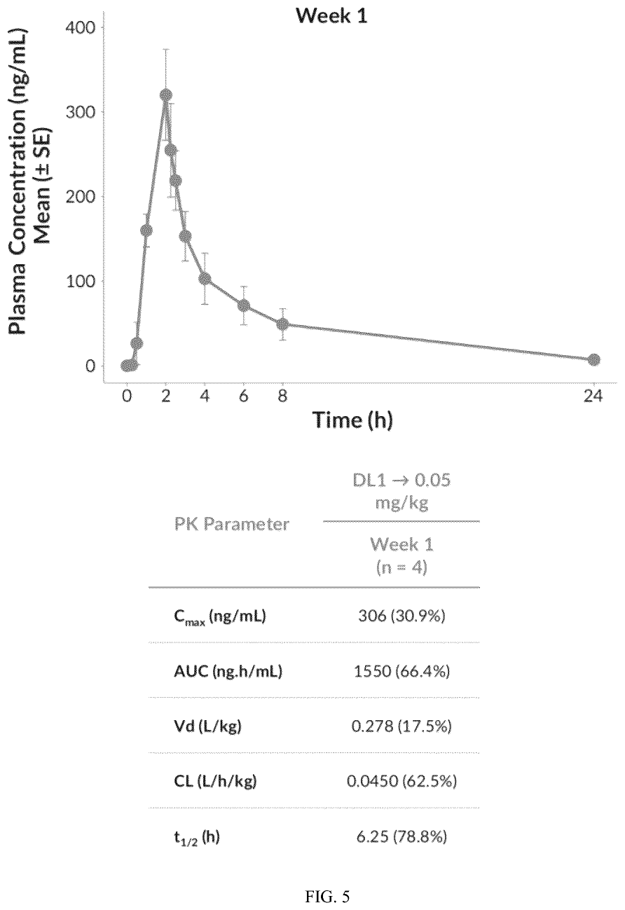

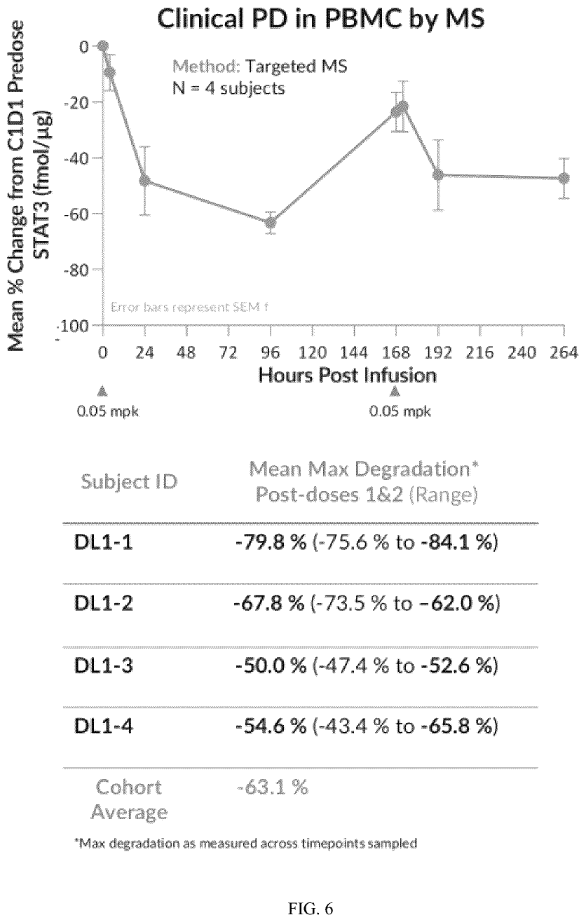

FIG. 5 shows PK data from 4 patients enrolled in DL1. FIG. 6 shows STAT3 degradation in blood at DL1. Observed STAT3 degradation of 50-80% in PBMCs at Dose Level 1 is consistent with the range predicted for tumor based on preclinical modeling of SUDHL1 xenograft PK-PD data. Maximal degradation is observed between 24-96 hours post infusion in Cycle 1 weeks 1 & 2, with recovery of STAT3 levels between doses, as seen in preclinical models.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A liquid formulation comprising Compound A, or a pharmaceutically acceptable salt thereof, and a pharmaceutically acceptable excipient and/or carrier:
wherein Compound A is (2-(((5S,8S,10aR)-3-acetyl-8-(((S)-5-amino-1-(2-chloro-3-(4-(((S)-1-((2S, 4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl) amino)-4-oxobutyl)phenoxy)-5-oxopentan-2-yl)carbamoyl)-6-oxodecahydropyrrolo [1,2-a][1,5]diazocin-5-yl)carbamoyl)-1H-indole-5-carbonyl)phosphonic acid.

2. The liquid formulation of claim 1, comprising Compound A at a concentration of about 0.995% w/w of the total weight of the formulation.

3. The liquid formulation of claim 1, comprising Compound A ammonium hydrogen salt at a concentration of about 1.00% w/w of the total weight of the formulation.

4. The liquid formulation of claim 1, comprising Compound A at a concentration of about 10 mg/mL.

5. The liquid formulation of claim 1, comprising Compound A ammonium hydrogen salt at a concentration of about 10.14 mg/mL.

6. The liquid formulation of any one of claims 1-5, comprising a sodium phosphate buffer at a concentration of about 50 mM.

7. The liquid formulation of any one of claims 1-5, comprising a sodium phosphate buffer at a concentration of about 0.64% w/w of the total weight of the formulation.

8. The liquid formulation of any one of claims 1-5, comprising a sodium phosphate buffer at a concentration of about 6.4 mg/mL.

9. The liquid formulation of any one of claims 1-8, which is at about pH 6.5.

10. The liquid formulation of any one of claims 1-9, which is a formulation selected from the following:
1) a liquid formulation at about pH 6.5, comprising Compound A at a concentration of about 0.995% w/w of the total weight of the formulation, and a sodium phosphate buffer at a concentration of about 50 mM;
2) a liquid formulation at about pH 6.5, comprising Compound A at a concentration of about 10 mg/mL, and a sodium phosphate buffer at a concentration of about 50 mM;
3) a liquid formulation at about pH 6.5, comprising Compound A at a concentration of about 0.995% w/w of the total weight of the formulation, and a sodium phosphate buffer at a concentration of about 0.64% w/w of the total weight of the formulation;
4) a liquid formulation at about pH 6.5, comprising Compound A at a concentration of about 10 mg/mL, and a sodium phosphate buffer at a concentration of about 0.64% w/w of the total weight of the formulation;
5) a liquid formulation at about pH 6.5, comprising Compound A at a concentration of about 0.995% w/w of the total weight of the formulation, and a sodium phosphate buffer at a concentration of about 6.4 mg/mL;
6) a liquid formulation at about pH 6.5, comprising Compound A at a concentration of about 10 mg/mL, and a sodium phosphate buffer at a concentration of about 6.4 mg/mL;
7) a liquid formulation at about pH 6.5, comprising Compound A ammonium hydrogen salt at a concentration of about 1.00% w/w of the total weight of the formulation, and a sodium phosphate buffer at a concentration of about 50 mM;
8) a liquid formulation at about pH 6.5, comprising Compound A ammonium hydrogen salt at a concentration of about 10.14 mg/mL, and a sodium phosphate buffer at a concentration of about 50 mM;
9) a liquid formulation at about pH 6.5, comprising Compound A ammonium hydrogen salt at a concentration of about 1.00% w/w of the total weight of the formulation, and a sodium phosphate buffer at a concentration of about 0.64% w/w of the total weight of the formulation;
10) a liquid formulation at about pH 6.5, comprising Compound A ammonium hydrogen salt at a concentration of about 10.14 mg/mL, and a sodium phosphate buffer at a concentration of about 0.64% w/w of the total weight of the formulation;
11) a liquid formulation at about pH 6.5, comprising Compound A ammonium hydrogen salt at a concentration of about 1.00% w/w of the total weight of the formulation, and a sodium phosphate buffer at a concentration of about 6.4 mg/mL; and
12) a liquid formulation at about pH 6.5, comprising Compound A ammonium hydrogen salt at a concentration of about 10.14 mg/mL, and a sodium phosphate buffer at a concentration of about 6.4 mg/mL.

11. The liquid formulation of any one of claims 1-10, which is a unit dosage form, with a volume of about 10 mL.

12. A method for treating a hematological malignancy or solid tumor in a patient, comprising administering to the patient a therapeutically effect amount of the liquid formulation of any one of claims 1-11.

13. The method of claim 12, wherein the hematological malignancy or solid tumor is a relapsed or refractory lymphoma.

14. The method of claim 12, wherein the hematological malignancy or solid tumor is selected from large granular lymphocytic leukemia (LGL-L), peripheral T-cell lymphoma (PTCL), and cutaneous T-cell lymphoma (CTCL).

15. The method of any one of claim 12-14, wherein the method comprises administering up to about 3.0 mg/kg of Compound A to the patient per day.

16. The method of any one of claim 12-14, wherein the method comprises administering up to about 500 mg of Compound A to the patient per day.

17. The method of any one of claims 12-16, wherein the method comprises administering Compound A to the patient intravenously.

18. The method of any one of claims 12-17, wherein the method comprises administering Compound A to the patient once per week (QW).

19. The method of any one of claims 12-18, wherein the method comprises administering Compound A to the patient on days 1, 8, 15, and 22 of a 28-day cycle.
